(19) United States
(12) Patent Application Publication
(10) Pub. No.: US 2016/0038485 A1
(43) Pub. Date: Feb. 11, 2016

(54) PHASE 2 INDUCERS AND RELATED SIGNALING PATHWAYS PROTECT CARTILAGE AGAINST INFLAMMATION, APOPTOSIS AND STRESS

(71) Applicant: THE JOHNS HOPKINS UNIVERSITY, Baltimore, MD (US)

(72) Inventors: Konstantinos Konstantopoulos, Ellicott City, MD (US); Paul Talalay, Baltimore, MD (US); Zachary R. Healy, Millersville, MD (US)

(21) Appl. No.: 14/868,589

(22) Filed: Sep. 29, 2015

Related U.S. Application Data
(63) Continuation of application No. 12/299,209, filed on Sep. 21, 2009, now abandoned.
(60) Provisional application No. 60/796,198, filed on May 1, 2006.

(51) Int. Cl. A61K 31/505; A61K 31/26; A61K 45/06; A61K 9/00

(52) U.S. Cl. CPC A61K 31/505; A61K 9/0014; A61K 31/26; A61K 45/06

(57) ABSTRACT

Disclosed herein are novel compounds, their use in the treatment and prevention of joint and/or cartilage inflammation that provide an alternative to the NSAIDS and selective COX-2 inhibitors by activating endogenous detoxifying cellular defense mechanisms that act to neutralize toxic cellular intermediate. These compounds are PPAR-alpha agonists and/or phase 2 gene activators.

### CROSS-REFERENCE TO RELATED APPLICATIONS

The application claims benefit of priority to U.S. Provisional Patent Application No. 60/796,198, filed May 1, 2006, which is incorporated in its entirety herein by reference.

### FIELD OF INVENTION

Disclosed herein are novel alternatives to the NSAIDS and COX-2 selective inhibitors to mitigate cartilagenous inflammation and pain by activating endogenous detoxifying cellular defense mechanisms that act to neutralize electrophilic reactive intermediates.

### BACKGROUND OF THE INVENTION

Arthritis is one of the leading causes of chronic disability in the United States and other developing countries. According to the Centers for Disease Control and Prevention (CDC), in 2001, arthritis and other rheumatic conditions effected 70 million adults and approximately 60% of those 65 years or older.

The arthridities are classified under the rheumatic diseases of the musculoskeletal system. Disorders involving the joints can be due to a primary disease process of articular tissue or a secondary manifestation of a systemic or multi-systemic disorder. Osteoarthritis (OA), also called degenerative joint disease (DJD), and Rheumatoid arthritis (RA) are two of the most common forms of arthritis. Biomechanical stress and inflammation are thought to play a role in the progressive loss of articular cartilage in OA, and one of the hallmarks of RA is progressive destruction of joint cartilage. Joint inflammation can also be a component in many of the other rheumatologic disorders such as systemic lupus erythematosus, reiter's syndrome, osteitis deformans, psoriatic arthritis, primary sjogrens syndrome, whipples disease, and the inflammatory bowel diseases, to name just a few.

Many of the arthritic conditions including OA and RA are chronic progressive disorders requiring long term treatment of associated pain and inflammation. Currently available non-invasive medical treatments rely heavily on anti-inflammatory agents that inhibit the cyclooxygenase (COX) enzyme, also known as prostaglandin H synthase, (PGHS). The COX enzyme catalyzes the production of the family of prostaglandins (PGs), widely accepted to be one class of inflammatory response mediators. However, the risk of significant gastrointestinal (GI) sequela from the non-selective COX inhibitors-the non-steroidal anti-inflammatory drugs, (NSAIDS), and the increased risk of myocardial infarction (heart attack) and stroke associated with the COX-2 selective inhibitors pose significant barriers to the use of these agents. Therefore, there is a need in the art for novel agents that bypass the direct method of action of the NSAIDS and COX-2 selective inhibitors and that act preferentially on articular tissue.

### BRIEF SUMMARY OF THE FIGURES

FIG. 1 Phenotype-specific effects of shear stress duration and intensity on phase 2 response. Human umbilical vein endothelial cells (HUVECs) and human chondrocyte cells (T/C28a2) were subjected to either static conditions or to laminar shear flow (5, 20, or 40 dyn/cm²) for 24 or 48 hours and NQO1 specific activities (A) and total glutathione levels (per mg protein) (B) were determined. Data are relative to static controls. Bars are mean±SEM (n=4-7, *p<0.01 and §p<0.05 with respect to static control). (C) ARE-driven NQO1 promoter activity in response to shear stress stimulation and phase 2 inducers, (left) T/C28a2 cells were transfected with pNQO1/ARE-luc vector and exposed to either static conditions or laminar shear flow (5 or 20 dyn/cm²) for 24 or 48 hours, (right) To determine the efficacy of phase 2 inducers, transfected cultures were treated with solvent (0.1%), SFN (1.25 μM) or D3T (5 μM) for 24 h under static conditions. ARE-driven firefly luciferase activity was normalized to Renilla luciferase and green fluorescent protein (GFP) expression. Data are relative to static controls (n=4, *p<0.01 and §p<0.05 with respect to static control).

FIG. 2 Effects of phase 2 induction on shear-dependent phase 2 response in chondrocytes. Cell were treated with DMSO (0.1%), sulforaphane (SFN) (1.25 μM), or D3T (5 μM) for 24 hours, subjected to either static conditions or laminar shear flow (20 or 40 dyn/cm²) for 24 or 48 hours in the presence of the agent, and NQO1 enzyme activity (A) and GSH levels (B) were determined. Data are relative to static controls (n=3-9, *p<0.01 with respect to shear stress-paired solvent-treated controls).

FIG. 3 Effects of the phase 2 inducer D3T on COX-2 protein levels in shear-activated chondrocytes. Cells, treated with either solvent (DMSO, 0.1%) or D3T (5 μM) were subjected to either static or laminar shear (5 or 20 dyn/cm²) for 48 hours in the presence of agent. Fluorescence intensity is proportional to COX-2 expression. COX-I expression remains unchanged (n=3).

FIG. 4 Effects of phase 2 gene induction on shear-induced COX-2-dependent prostaglandin E2 (PGE2) production in chondrocytes. Cells were treated with either solvent (0.1% DMSO) or D3T (5 μM) or transfected with pCMV-null or pCMV-mNrf2 (24 hours), and then exposed to fluid shear (48 hours). PGE2 levels were determined in culture media at the indicated times. Data are relative to paired static controls at t=0 (n=4, transfection efficiency=32.8±4.5%).

FIG. 5 Effects of inhibition of COX-2 activity on shear-dependent phase 2 response in chondrocytes. Cells were treated with CAY 10404 (6.75 μM) or control solvent (0.1% DMSO) for 2 hours, exposed to the static conditions or laminar shear flow (20 dyn/cm²) for 48 hours in the presence of agent, and NQO1 enzyme activities (A) and total GSH levels (B) were determined. Data obtained with NS398 (30 μM) were indistinguishable from those with CAY10404. Data are relative to static controls (n=3-9, *, P<0.01 with respect to static controls).

FIG. 6 Effects of phase 2 inducers and COX-2 inhibitors on shear-mediated DNA-fragmentation, mitochondrial membrane permeabilization, and caspase-9 protein levels. T/C-28a2 cells were treated with the solvent (DMSO) or D3T (5 μM) for 24 hours or solvent CAY10404 (6.75 μM) or NS298 (30 μM) for 2 hours, and then exposed to either static or laminar flow (20 dyn/cm²) for 48 hours in the presence of the agent. Cells were examined for markers of apoptosis by using DNA fragmentation (TUNEL, A), mitochondrial membrane permeabilization (MMP, B) and caspase-9 expression (C).

FIG. 7 Effects of PI3K activity on shear-dependent phase 2 response in chondrocytes. Cell were transfected with pBJ M>>pl 10* (constitutively active PI3-K), pBJ M>>pl 10>>UR (Δkinase mutant), or pBJ-null vector, subjected to static conditions or laminar flow (20 dyn/cm2) for 48 hours, and NQO1 enzyme activity (A), and total glutathione (GSH) levels (B) were determined. Data are relative to null transfected static cultures (n=4, *p<0.01 with respect to the static control, J, p<0.05 with respect to null-transfected shear).

FIG. 8 Temporal effects of shear stress on PPAR mRNA expression in human chondrocytic cells. T/C-28a2 chondrocytes were exposed to either static conditions or laminar fluid shear (20 dyn/cm2) for prescribed shear exposure times. Transcript (mRNA) expression ratios (shear/static) for PPAR genes were determined by cDNA microarray analysis and confirmed by qRT-PCR (n=3-4).

FIG. 9 Effects of PPARα receptor modulation on anti- and proinflammatory signaling in shear-activated human chondrocytic cells. T/C-28a2 cells were treated with the specific PPARα ligand, WyI 4643 (10 μM), for 2 h before being subjected to a shear stress level of 20 dyn/cm² for 48 h in the presence of WyI 4643. Total RNA was extracted from sheared and static specimens, and mRNA expression ratios (shear/static) for genes involved in the phase 2 response (left panels) and COX-2 activation (right panel) were determined by cDNA microarray analysis and confirmed by qRT-PCR (n=3).

FIG. 10 Effects of COX-2 selective inhibitors CAY 10404 and NS398 on shear-induced PPAR mRNA expression in human chondrocytic cells. T/C-28a2 cells were treated with solvent (0.1% DMSO), CAY10404 (5 μM), or NS398 (30 μM), and exposed to either static conditions or laminar fluid shear (20 dyn/cm²) for 48 h in the presence of the agent. Transcript (mRNA) expression ratios (shear/static) for PPAR genes were determined by cDNA microarray analysis and confirmed by qRT-PCR (n=3-4).

### SUMMARY OF THE INVENTION

One embodiment of the invention is a composition comprising a selective PPARα agonist and a phase 2 gene activator.

Another embodiment of the invention is a composition comprising a selective PPARα agonist.

Yet another embodiment of the invention is a lotion, cream, foam or gel for treating joint or cartilaginous inflammation or pain comprising a therapeutically effective amount of a phase 2 gene activator and one or more additives and adjuvants that are common in the art of cosmetic and/or medical compositions for topical application in a mammal.

One embodiment of the invention is a composition comprising a selective PPARα agonist and a phase 2 gene activator or a composition comprising a lotion, cream, foam or gel for treating or preventing joint or cartilaginous inflammation or pain comprising a therapeutically effective amount of a phase 2 gene activator wherein said phase 2 gene activator comprises a glucosinolate or isothyocyanate.

Another embodiment of the invention is a composition comprising a selective PPARα agonist and a phase 2 gene activator or a composition comprising a lotion, cream, foam or gel for treating or preventing joint or cartilaginous inflammation or pain comprising a therapeutically effective amount of a phase 2 gene activator wherein said phase 2 gene activator comprises a glucosinolate wherein the glucosinolate is selected from one or more of said glucosinolates listed in table 1.

Still, another embodiment of the invention is a composition comprising a selective PPARα agonist and a phase 2 gene activator or a composition comprising a lotion, cream, foam or gel for treating or preventing joint or cartilaginous inflammation or pain comprising a therapeutically effective amount of a phase 2 gene activator wherein said phase 2 gene activator comprises a isothiocyanate wherein the said isothiocyanate is sulforaphane and/or one or more of said sulforaphane analogs listed in table 2.

Yet still, another embodiment of the invention is a composition comprising a selective PPARα agonist and a phase 2 gene activator or a composition comprising a selective PPARα agonist wherein the selective PPARα is selected from the group consisting of Wy14643, clofibrate, fenofibrate, 8(S)-Hydroxy-(5Z,9E,HZ,14Z)-eicosatetraenoic acid (8(S)-HETE), leukotriene B4 (LTB4), tetradecythioacetic acid (TTA), GW 9578, and GW 7647.

One embodiment of the invention is a method for treating joint or cartilaginous inflammation, comprising administering a therapeutically effective amount of a selective PPARα agonist and a phase 2 gene activator in a mammal.

Another embodiment of the invention is a method for preventing joint or cartilaginous inflammation, comprising administering a therapeutically effective amount of a selective PPARα agonist and a phase 2 gene activator in a mammal.

Still, another embodiment of the invention is a method for treating joint or cartilaginous inflammation comprising administering a therapeutically effective amount of a selective PPARα agonist in a mammal.

Yet, another embodiment of the invention is a method for preventing joint or cartilaginous inflammation comprising administering a therapeutically effective amount of a selective PPARα agonist in a mammal.

A further embodiment of the invention is a method for treating joint or cartilaginous inflammation comprising administering a lotion, cream, foam or gel comprising a therapeutically effective amount of a phase 2 gene activator and one or more additives and adjuvants that are common in the art of cosmetic and/or medical compositions for topical application in a mammal.

Yet a further embodiment of the invention is a method for preventing joint or cartilaginous inflammation comprising administering a lotion, cream, foam or gel comprising a therapeutically effective amount of a phase 2 gene activator and one or more additives and adjuvants that are common in the art of cosmetic and/or medical compositions for topical application in a mammal.

One embodiment of the invention is a method for treating joint or cartilaginous pain comprising administering a therapeutically effective amount of a selective PPARα agonist and a phase 2 gene activator in a mammal.

Another embodiment of the invention is a method for preventing joint or cartilaginous pain comprising administering a therapeutically effective amount of a selective PPARα agonist and a phase 2 gene activator in a mammal.

Still another embodiment of the invention is a method for treating joint or cartilaginous pain comprising administering a therapeutically effective amount of a selective PPARα agonist in a mammal.

Yet another embodiment of the invention is a method for preventing joint or cartilaginous pain comprising administering a therapeutically effective amount of a selective PPARα agonist in a mammal.

An additional embodiment of the invention is a method for treating joint or cartilaginous pain comprising administering a lotion, cream, foam or gel comprising a therapeutically effective amount of a phase 2 gene activator and one or more additives and adjuvants that are common in the art of cosmetic and/or medical compositions for topical application in a mammal.

Still another embodiment of the invention is a method for preventing joint or cartilaginous pain comprising administering a lotion, cream, foam or gel comprising a therapeutically effective amount of a phase 2 gene activator and one or more additives and adjuvants that are common in the art of cosmetic and/or medical compositions for topical application in a mammal.

One embodiment of the invention is a method for treating intra-operative joint or cartilaginous inflammation comprising administering a therapeutically effective amount of a selective PPARα agonist and a phase 2 gene activator in a mammal.

Another embodiment of the invention is a method for preventing intraoperative joint or cartilaginous inflammation comprising administering a therapeutically effective amount of a selective PPARα agonist and a phase 2 gene activator in a mammal.

Yet another embodiment of the invention is a method for treating intraoperative joint or cartilaginous inflammation comprising administering a therapeutically effective amount of a selective PPARα agonist in a mammal.

Still, an additional embodiment of the invention is a method for preventing intra-operative joint or cartilaginous inflammation comprising administering a therapeutically effective amount of a selective PPARα agonist in a mammal.

One embodiment of the invention is a method for treating post-operative joint or cartilaginous inflammation comprising administering a therapeutically effective amount of a selective PPARα agonist and a phase 2 gene activator in a mammal.

Another embodiment of the invention is a method for preventing postoperative joint or cartilaginous inflammation comprising administering a therapeutically effective amount of a selective PPARα agonist and a phase 2 gene activator in a mammal.

Still another embodiment of the invention is a method for treating postoperative joint or cartilaginous inflammation comprising administering a therapeutically effective amount of a selective PPARα agonist in a mammal.

An additional embodiment of the invention is a method for preventing postoperative joint or cartilaginous inflammation comprising administering a therapeutically effective amount of a selective PPARα agonist in a mammal.

A further embodiment of the invention is a method for treating postoperative joint or cartilaginous pain comprising administering a therapeutically effective amount of a selective PPARα agonist and a phase 2 gene activator in a mammal.

Yet still, another embodiment of the invention is a method for preventing post-operative joint or cartilaginous pain comprising administering a therapeutically effective amount of a selective PPARα agonist and a phase 2 gene activator in a mammal.

Another embodiment of the invention is a method for treating post-operative joint or cartilaginous pain comprising administering a therapeutically effective amount of a selective PPARα agonist in a mammal.

Yet another embodiment of the invention is a method for preventing postoperative joint or cartilaginous pain comprising administering a therapeutically effective amount of a selective PPARα agonist in a mammal.

A further embodiment of the invention is a method for treating postoperative joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal.

One embodiment of the invention is a diagnostic method for distinguishing between costal cartilage inflammation (costochondritis) and pain from non-costal cartilage (non-costochondritis) inflammation and pain comprising administering a therapeutically effective amount of a selective PPARα agonist and a phase 2 gene activator in a mammal.

Another embodiment of the invention is a diagnostic method for distinguishing between costal cartilage inflammation (costochondritis) and pain from non-costal cartilage (non-costochondritis) inflammation and pain comprising administering a therapeutically effective amount of a selective PPARα agonist in a mammal.

Still, another embodiment of the invention is a method for treating or preventing joint or cartilaginous inflammation or pain, comprising administering a therapeutically effective amount of a selective PPARα agonist and a phase 2 gene activator in a mammal or a method for treating or preventing joint or cartilaginous inflammation or pain comprising administering a therapeutically effective amount of a selective PPARα agonist in a mammal or a method for treating or preventing intraoperative joint or cartilaginous inflammation comprising administering a therapeutically effective amount of a selective PPARα agonist and a phase 2 gene activator in a mammal or method for treating or preventing intra-operative joint or cartilaginous inflammation comprising administering a therapeutically effective amount of a selective PPARα agonist in a mammal or method for treating or preventing post-operative joint or cartilaginous inflammation or pain comprising administering a therapeutically effective amount of a selective PPARα agonist and a phase 2 gene activator in a mammal or a method for treating or preventing postoperative joint or cartilaginous inflammation or pain comprising administering a therapeutically effective amount of a selective PPARα agonist in a mammal wherein said PPARα is selected from the group consisting of WyI 4643, clofibrate, fenofibrate, 8(S)-Hydroxy-(5Z,9E,1IZ,14Z)-eicosatetraenoic acid (8(S)-HETE), leukotriene B4 (LTB4), tetradecythioacetic acid (TTA), GW 9578 and GW 7647.

An additional embodiment of the invention is a diagnostic method for distinguishing between costal cartilage inflammation (costochondritis) and pain from non-costal cartilage (non-costochondritis) inflammation and pain comprising administering a therapeutically effective amount of a selective PPARα agonist and a phase 2 gene activator in a mammal or a diagnostic method for distinguishing between costal cartilage inflammation (costochondritis) and pain from non-costal cartilage (non-costochondritis) inflammation and pain comprising administering a therapeutically effective amount of a selective PPARα agonist in a mammal wherein said PPARα is selected from the group consisting of WyI 4643, clofibrate, fenofibrate, 8(S)-Hydroxy-(5Z,9E,1IZ,14Z)-eicosatetraenoic acid (8(S)-HETE), leukotriene B4 (LTB4), tetradecythioacetic acid (TTA), GW 9578 and GW 7647.

One embodiment of the in invention is a method for treating or preventing joint or cartilaginous inflammation or pain, comprising administering a therapeutically effective amount of a selective PPARα agonist and a phase 2 gene activator in a mammal or a method for treating or preventing joint or cartilaginous inflammation or pain comprising administering a therapeutically effective amount of a selective PPARα agonist in a mammal or a method for treating or preventing intraoperative joint or cartilaginous inflammation comprising administering a therapeutically effective amount of a selective PPARα agonist and a phase 2 gene activator in a mammal or method for treating or preventing intra-operative joint or cartilaginous inflammation comprising administering a therapeutically effective amount of a selective PPARα agonist in a mammal or method for treating or preventing post-operative joint or cartilaginous inflammation or pain comprising administering a therapeutically effective amount of a selective PPARα agonist and a phase 2 gene activator in a mammal or a method for treating or preventing postoperative joint or cartilaginous inflammation or pain comprising administering a therapeutically effective amount of a selective PPARα agonist in a mammal wherein said selective PPARα is selected from the group consisting of WyI 4643, clofibrate, fenofibrate, 8(S)-Hydroxy-(5Z,9E,HZ,14Z)-eicosatetraenoic acid (8(S)-HETE), leukotriene B4 (LTB4), tetradecythioacetic acid (TTA), GW 9578, and GW 7647 wherein preferably 0.05-150 mg/kg/day, more preferably 0.1-15 mg/kg/day, even more preferably 0.05-4.0 mg/kg/day of said selective PPARα agonist is administered in a mammal.

Yet another embodiment of the invention is a diagnostic method for distinguishing between costal cartilage inflammation (costochondritis) and pain from non-costal cartilage (non-costochondritis) inflammation and pain comprising administering a therapeutically effective amount of a selective PPARα agonist and a phase 2 gene activator in a mammal or a diagnostic method for distinguishing between costal cartilage inflammation (costochondritis) and pain from non-costal cartilage (non-costochondritis) inflammation and pain comprising administering a therapeutically effective amount of a selective PPARα agonist in a mammal wherein said PPARα is selected from the group consisting of WyI 4643, clofibrate, fenofibrate, 8(S)-Hydroxy-(5Z,9E,1IZ,14Z)-eicosatetraenoic acid (8(S)-HETE), leukotriene B4 (LTB4), tetradecythioacetic acid (TTA), GW 9578, and GW 7647 wherein preferably 0.05-150 mg/kg/day, more preferably 0.1-15 mg/kg/day, even more preferably 0.05-4.0 mg/kg/day of said selective PPARα agonist is administered in a mammal.

An additional embodiment of the invention is a method for treating or preventing joint or cartilaginous inflammation or pain, comprising administering a therapeutically effective amount of a selective PPARα agonist and a phase 2 gene activator in a mammal or a method for treating or preventing joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal, or a method for treating or preventing intra-operative joint or cartilaginous inflammation comprising administering a therapeutically effective amount of a selective PPARα agonist and a phase 2 gene activator in a mammal or a method for treating or preventing postoperative joint or cartilaginous inflammation or pain comprising administering a therapeutically effective amount of a selective PPARα agonist and a phase 2 gene activator in a mammal, or a method for treating post-operative joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal, or a diagnostic method for distinguishing between costal cartilage inflammation (costochondritis) and pain from non-costal cartilage (non-costochondritis) inflammation and pain comprising administering a therapeutically effective amount of a selective PPARα agonist and a phase 2 gene activator in a mammal wherein said phase 2 gene activator comprises a glucosinolate or isothyocyanate.

One embodiment of the invention is a method for treating or preventing joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal, or a method for treating post-operative joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal wherein said phase 2 gene activator comprises a glucosinolate or isothiocyanate.

A further embodiment of the invention is a method for treating or preventing joint or cartilaginous inflammation or pain, comprising administering a therapeutically effective amount of a selective PPARα agonist and a phase 2 gene activator in a mammal or a method for treating or preventing joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal, or a method for treating or preventing intra-operative joint or cartilaginous inflammation comprising administering a therapeutically effective amount of a selective PPARα agonist and a phase 2 gene activator in a mammal, or a method for treating or preventing postoperative joint or cartilaginous inflammation or pain comprising administering a therapeutically effective amount of a selective PPARα agonist and a phase 2 gene activator in a mammal, or a method for treating post-operative joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal, or a diagnostic method for distinguishing between costal cartilage inflammation (costochondritis) and pain from non-costal cartilage (non-costochondritis) inflammation and pain comprising administering a therapeutically effective amount of a selective PPARα agonist and a phase 2 gene activator in a mammal wherein said phase 2 gene activator comprises a glucosinolate or isothyocyanate wherein said glucosinolate is selected from one or more of said glucosinolates listed in table 1.

Yet another embodiment of the invention is a method for treating or preventing joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal, or a method for treating post-operative joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal wherein said phase 2 gene activator comprises a glucosinolate or isothiocyanate, wherein said glucosinolate is selected from one or more of said glucosinolates listed in table 1.

Another embodiment of the invention is a method for treating or preventing joint or cartilaginous inflammation or pain, comprising administering a therapeutically effective amount of a selective PPARα agonist and a phase 2 gene activator in a mammal or a method for treating or preventing joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal or a method for treating or preventing intra-operative joint or cartilaginous inflammation comprising administering a therapeutically effective amount of a selective PPARα agonist and a phase 2 gene activator in a mammal or a method for treating or preventing postoperative joint or cartilaginous inflammation or pain comprising administering a dierapeutically effective amount of a selective PPARα agonist and a phase 2 gene activator in a mammal, or a method for treating post-operative joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal, or a diagnostic method for distinguishing between costal cartilage inflammation (costochondritis) and pain from non-costal cartilage (non-costochondritis) inflammation and pain comprising administering a therapeutically effective amount of a selective PPARα agonist and a phase 2 gene activator in a mammal wherein said phase 2 gene activator comprises a glucosinolate or isothyocyanate wherein said glucosinolate is selected from one or more of said glucosinolates listed in table 1 wherein preferably, 1-50 mg/kg/day, more preferably 2-20 mg/kg/day, even more preferably 2-10 mg/kg/day of said glucosinolate listed in table 1 is administered to a mammal.

Another embodiment of the invention is a method for treating or preventing joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal, or a method for treating post-operative joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal wherein said phase 2 gene activator comprises a glucosinolate or isothiocyanate, wherein preferably, 0.1-100 mM, more preferably 1-50 mM, even more preferably 0.1-10 mM of said glucosinolate listed in table 1 is administered to the skin overlying a joint or cartilage.

Yet another embodiment of the invention is a method for treating or preventing joint or cartilaginous inflammation or pain, comprising administering a therapeutically effective amount of a selective PPARα agonist and a phase 2 gene activator in a mammal or a method for treating or preventing joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal, or a method for treating or preventing intra-operative joint or cartilaginous inflammation comprising administering a therapeutically effective amount of a selective PPARα agonist and a phase 2 gene activator in a mammal or a method for treating or preventing postoperative joint or cartilaginous inflammation or pain comprising administering a therapeutically effective amount of a selective PPARα agonist and a phase 2 gene activator in a mammal or a method for treating post-operative joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal or a diagnostic method for distinguishing between costal cartilage inflammation (costochondritis) and pain from non-costal cartilage (non-costochondritis) inflammation and pain comprising administering a therapeutically effective amount of a selective PPARα agonist and a phase 2 gene activator in a mammal wherein said phase 2 gene activator comprises a glucosinolate or isothyocyanate wherein said isothiocyanate is sulforaphane and/or one or more of said sulforaphane analogs listed in table 2.

One embodiment of the invention is a method for treating or preventing joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal, or a method for treating post-operative joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal wherein said phase 2 gene activator comprises a glucosinolate or isothiocyanate, wherein said isothiocyanate is a sulforaphane and/or one or more of said sulforaphane analogs listed in table 2.

Yet another embodiment of the invention is a method for treating or preventing joint or cartilaginous inflammation or pain, comprising administering a therapeutically effective amount of a selective PPARα agonist and a phase 2 gene activator in a mammal, or a method for treating or preventing joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal, or a method for treating or preventing intra-operative joint or cartilaginous inflammation comprising administering a therapeutically effective amount of a selective PPARα agonist and a phase 2 gene activator in a mammal or a method for treating or preventing postoperative joint or cartilaginous inflammation or pain comprising administering a therapeutically effective amount of a selective PPARα agonist and a phase 2 gene activator in a mammal or a method for treating post-operative joint or cartilaginous inflammation or pain comprising administering a lotion, cream, foam or gel for comprising a therapeutically effective amount of a phase 2 gene activator and one or more additives and adjuvants such as lipophilic or hydrophilic gelling agents, preservatives, fillers and other agents that are common in art of compositions for topical application in a mammal or a diagnostic method for distinguishing between costal cartilage inflammation (costochondritis) and pain from non-costal cartilage (non-costochondritis) inflammation and pain comprising administering a therapeutically effective amount of a selective PPARα agonist and a phase 2 gene activator in a mammal wherein said phase 2 gene activator comprises a glucosinolate or isothyocyanate wherein said isothiocyanate is sulforaphane and/or one or more of said sulforaphane analogs listed in table 2 wherein preferably 1-150 mg/kg/day, more preferably 1-75 mg/kg/day, even more preferably 5-50 mg/kg/day of said sulforaphane and/or one or more said sulforaphane analogs listed in table 2 are administered to a mammal.

Another embodiment of the invention is a method for treating or preventing joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal, or a method for treating post-operative joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal wherein said phase 2 gene activator comprises a glucosinolate or isothiocyanate, wherein said glucosinolate is selected from one or more of said glucosinolates listed in table 1, wherein preferably, 0.1-100 mM, more preferably 1-50 mM, even more preferably 0.1-10 mM of said sulforaphane and/or one or more said sulforaphane analogs listed in table 2 are administered to the skin overlying a joint or cartilage.

One embodiment of the invention is a method for treating or preventing joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal or a for treating postoperative joint or cartilaginous inflammation or pain comprising administering a lotion, cream, foam or gel comprising a therapeutically effective amount of a phase 2 gene activator and one or more additives and adjuvants that are common in the art of cosmetic and/or medical compositions for topical application in a mammal wherein said administration is topical.

Another embodiment of the invention is a method for treating or preventing joint or cartilaginous inflammation or pain, comprising administering a therapeutically effective amount of a selective PPARα agonist and a phase 2 gene activator in a mammal, or a method for treating or preventing joint or cartilaginous inflammation or pain comprising administering a therapeutically effective amount of a selective PPARα agonist in a mammal wherein said administration is oral, topical, parenteral, via a gastrointestinal tube, or by injection.

Still another embodiment of the invention is a method for treating or preventing joint or cartilaginous inflammation or pain, comprising administering a therapeutically effective amount of a selective PPARα agonist and a phase 2 gene activator in a mammal or a method for treating or preventing joint or cartilaginous inflammation or pain comprising administering a therapeutically effective amount of a selective PPARα agonist in a mammal wherein said administration is oral.

Yet another embodiment of the invention is a method for treating or preventing joint or cartilaginous inflammation or pain, comprising administering a therapeutically effective amount of a selective PPARα agonist and a phase 2 gene activator in a mammal, or a method for treating or preventing joint or cartilaginous inflammation or pain comprising administering a therapeutically effective amount of a selective PPARα agonist in a mammal, wherein said administration is topical.

A further embodiment of the invention is a method for treating or preventing joint or cartilaginous inflammation or pain, comprising administering a therapeutically effective amount of a selective PPARα agonist and a phase 2 gene activator in a mammal or a method for treating or preventing joint or cartilaginous inflammation or pain comprising administering a therapeutically effective amount of a selective PPARα agonist in a mammal wherein said administration is parenteral.

An additional embodiment of the invention is a method for treating or preventing joint or cartilaginous inflammation or pain, comprising administering a therapeutically effective amount of a selective PPARα agonist and a phase 2 gene activator in a mammal or a method for treating or preventing joint or cartilaginous inflammation or pain comprising administering a therapeutically effective amount of a selective PPARα agonist in a mammal wherein said administration is via a gastrointestinal tube.

Another embodiment of the invention is a method for treating or preventing joint or cartilaginous inflammation or pain, comprising administering a therapeutically effective amount of a selective PPARα agonist and a phase 2 gene activator in a mammal or a method for treating or preventing joint or cartilaginous inflammation or pain comprising administering a therapeutically effective amount of a selective PPARα agonist in a mammal wherein said administration is by injection.

One embodiment of the invention is a method for preventing post-operative joint or cartilaginous inflammation or pain comprising administering a therapeutically effective amount of a selective PPARα agonist and a phase 2 gene activator in a mammal or a method for preventing post-operative joint or cartilaginous inflammation or pain comprising administering a therapeutically effective amount of a selective PPARα agonist in a mammal, wherein said administration is preoperative, intraoperative, post-operative or any combination thereof.

Another embodiment of the invention is a method for preventing intraoperative joint or cartilaginous inflammation comprising administering a therapeutically effective amount of a selective PPARα agonist and a phase 2 gene activator in a mammal or a method for treating intraoperative joint or cartilaginous inflammation comprising administering a therapeutically effective amount of a selective PPARα agonist in a mammal wherein said administration is preoperative, intraoperative or both.

Yet another embodiment of the invention is a method for preventing postoperative joint or cartilaginous inflammation or pain comprising administering a therapeutically effective amount of a selective PPARα agonist and a phase 2 gene activator in a mammal or a method for preventing post-operative joint or cartilaginous inflammation or pain comprising administering a therapeutically effective amount of a selective PPARα agonist in a mammal, or a method for preventing intraoperative joint or cartilaginous inflammation comprising administering a therapeutically effective amount of a selective PPARα agonist and a phase 2 gene activator in a mammal or a method for treating intra-operative joint or cartilaginous inflammation comprising administering a therapeutically effective amount of a selective PPARα agonist in a mammal wherein said administration is preoperative.

Still yet another embodiment of the invention is a method for treating intraoperative joint or cartilaginous inflammation comprising administering a therapeutically effective amount of a selective PPARα agonist and a phase 2 gene activator in a mammal or a method for treating intra-operative joint or cartilaginous inflammation comprising administering a therapeutically effective amount of a selective PPARα agonist in a mammal, or a method for preventing post-operative joint or cartilaginous inflammation or pain comprising administering a therapeutically effective amount of a selective PPARα agonist and a phase 2 gene activator in a mammal or a method for preventing post-operative joint or cartilaginous inflammation or pain comprising administering a therapeutically effective amount of a selective PPARα agonist in a mammal or a method for preventing intra-operative joint or cartilaginous inflammation comprising administering a therapeutically effective amount of a selective PPARα agonist and a phase 2 gene activator in a mammal or a method for treating intra-operative joint or cartilaginous inflammation comprising administering a therapeutically effective amount of a selective PPARα agonist in a mammal wherein said administration is intra-operative.

A further embodiment of the invention is a method for treating postoperative joint or cartilaginous inflammation or pain comprising administering a therapeutically effective amount of a selective PPARα agonist and a phase 2 gene activator in a mammal or a method for treating post-operative joint or cartilaginous inflammation or pain comprising administering a therapeutically effective amount of a selective PPARα agonist in a mammal or a method for treating post-operative joint or cartilaginous inflammation or pain comprising administering a lotion, cream, foam or gel comprising a therapeutically effective amount of a phase 2 gene activator and one or more additives and adjuvants such as lipophilic or hydrophilic gelling agents, preservatives, fillers and other agents that are common in art of compositions for topical application in a mammal or a method for preventing post-operative joint or cartilaginous inflammation or pain comprising administering a therapeutically effective amount of a selective PPARα agonist and a phase 2 gene activator in a mammal or a method for preventing post-operative joint or cartilaginous inflammation or pain comprising administering a therapeutically effective amount of a selective PPARα agonist in a mammal or a method for preventing intra-operative joint or cartilaginous inflammation comprising administering a therapeutically effective amount of a selective PPARα agonist and a phase 2 gene activator in a mammal or a method for treating intra-operative joint or cartilaginous inflammation comprising administering a therapeutically effective amount of a selective PPARα agonist in a mammal wherein said administration is post-operative.

An additional embodiment of the invention is a method for preventing postoperative joint or cartilaginous inflammation or pain comprising administering a therapeutically effective amount of a selective PPARα agonist and a phase 2 gene activator in a mammal or a method for preventing post-operative joint or cartilaginous inflammation or pain comprising administering a therapeutically effective amount of a selective PPARα agonist in a mammal or a method for preventing intra-operative joint or cartilaginous inflammation comprising administering a therapeutically effective amount of a selective PPARα agonist and a phase 2 gene activator in a mammal or a method for treating intra-operative joint or cartilaginous inflammation comprising administering a therapeutically effective amount of a selective PPARα agonist in a mammal wherein said administration is pre-operative and intra-operative.

One embodiment of the invention is a method for preventing post-operative joint or cartilaginous inflammation or pain comprising administering a therapeutically effective amount of a selective PPARα agonist and a phase 2 gene activator in a mammal or a method for preventing post-operative joint or cartilaginous inflammation or pain comprising administering a therapeutically effective amount of a selective PPARα agonist in a mammal said administration is pre-operative and postoperative.

Another embodiment of the invention is a method for preventing postoperative joint or cartilaginous inflammation or pain comprising administering a therapeutically effective amount of a selective PPARα agonist and a phase 2 gene activator in a mammal or a method for preventing post-operative joint or cartilaginous inflammation or pain comprising administering a therapeutically effective amount of a selective PPARα agonist in a mammal wherein said administration is intra-operative and post operative.

Yet another embodiment of the invention is a method for preventing postoperative joint or cartilaginous inflammation or pain comprising administering a therapeutically effective amount of a selective PPARα agonist and a phase 2 gene activator in a mammal or a method for preventing post-operative joint or cartilaginous inflammation or pain comprising administering a therapeutically effective amount of a selective PPARα agonist in a mammal wherein said administration is pre-operative, intra-operative, and post-operative.

One embodiment of the invention is wherein the method of treating or preventing inflammation or pain in said mammal is a human mammal.

Another embodiment of the invention is wherein the method of treating or preventing inflammation or pain said mammal comprises a domesticated animal, a farm animal, an experimental animal or a commercial animal.

A further embodiment of the invention is wherein the said joint or cartilage being treated for inflammation or pain or inflammation or pain is being prevented is a joint of the hip, a knee, an ankle, a shoulder, an elbow, a wrist or a joint of a foot, a joint of a hand, or a joint of the spine.

Yet another embodiment of the invention is wherein the said joint or cartilage being treated or prophylaxed for inflammation or pain is the temporomandibular joint.

Still another embodiment of the invention is wherein the said joint or cartilage being treated or prophylaxed for inflammation or pain is the costal cartilage

One embodiment of the invention is a kit, comprising of a composition, comprising a selective PPARα agonist and a phase 2 gene activator or a composition, comprising a selective PPARα agonist, a needle and a syringe.

Another embodiment of the invention is a kit comprising of a composition, comprising a selective PPARα agonist and a phase 2 gene activator or a composition, comprising a selective PPARα agonist, a needle and a syringe for self-administration.

Yet another embodiment of the invention is a kit comprising of a composition, comprising a selective PPARα agonist and a phase 2 gene activator or a composition, comprising a selective PPARα agonist, a needle and a syringe for administration by another.

### DETAILED DESCRIPTION OF THE INVENTION

This invention teaches novel compositions of PPARα specific ligands and phase 2 gene activators alone or in combination for treating and preventing inflammation and pain preferentially in joints and chondrocytic tissue, thereby providing an alternative therapy to the non-selective and selective COX-2 inhibitors for joint inflammation and pain.

“Additives and adjuvants” herein refers to compositions that are known in the art of cosmetic and/or medical compositions and encompasses hydrophilic or lipophilic gelling agents, preservatives, antioxidants, solvents, fragrances, fillers, dyestuffs and colorants.

“Cartilage” herein refers to the specialized connective tissue comprising mature and/or young cartilage cells, adult chondrocytes and chondroblasts and the matrix of amorphous ground substance that surrounds a network of collagen fibers (Churchill's Medical Dictionary). Cartilage hereby includes the cartilagenous tissue that participates in synovial and non-synovial junctions and also to cartilage of the thorasic wall, the larynx, the trachea, the bronchi, and the nose and ears.

“Diagnostic method” herein refers to a method that contributes to the ability of one skilled in the art to discriminate between one probable cause of a symptom from another. For example, to distinguish chest pain that is primarily due to costochondritis (cartilage inflammation) from chest pain due to a non-costochondritis etiology.

“Gastrointestinal tube” or GI tube herein encompasses all the types of tubes that are used to access the gastrointestinal tract regardless of the medical purpose for which the tube is placed, such as those placed for enteral feeding or medication delivery (PEG and NG tubes), for lavage (washing), for determining GI contents, or for controlling GI bleeding.

“Injection” herein refers to the method of delivering the agent/compound by a means other than to the surface of the skin and includes delivery to the intraarticular space and related tissue, subcutaneous tissue, to the muscle, into the veins, into the vagina, or rectum.

“Joint” herein refers to the anatomical structure that connects at least two elements of anatomy and includes synovial and non-synovial junctions.

“Mammal” herein refers to human and non-human mammals.

“Human mammal” herein refers to all ages of humans from new born to the elderly and encompasses all genders.

“Domesticated mammal” herein refers to any mammal that lives and associates with humans and is most typically represented as a dog or a cat but herein encompasses any mammal though non-typically associated with humans is associating with humans. An example of the latter is the wolf.

“Farm mammal” herein refers to mammals associated with farms regardless of whether that animal is living on a farm and includes horses, cows, goats, sheep, pigs and others.

“Experimental animal” herein refers to animals used for scientific/investigational purposes such as primates, dogs, cats, pigs, rats, mice and others.

“Commercial animal” herein refers to animals that are utilized either transiently or habitually for profit. For example, this group includes cows bred to produce milk or for beef, competitive animals such as race horses and dogs and show animals such as show dogs, as well as all zoological mammals.

“Oral” herein includes any form of delivery of an agent/compound wherein the agent/compound is placed directly or indirectly into or through the nasal-oral cavity of the subject whether or not the agent/compound is swallowed. The term “oral” hereby includes sublingual, buccal, esophageal administration as well as delivery of the agent/compound through a nasogastric tube.

“Parenteral” herein includes any form of delivery of an agent/compound by a means other than by the mouth, such as delivery through the vein, into the muscle, into the intra-articular space and associated tissue, to the subcutaneous tissue, into the nasal cavity, vaginal canal or rectum.

“Selective PPARα agonist” herein denotes a PPARα agonist that has at least 5-fold greater affinity for PPARα than for PPARβ/δ or PPARγ.

“Therapeutically effective amount” herein refers to that amount of agent/compound that is sufficient to decrease or alleviate symptoms of inflammation and/or pain or is sufficient to decrease the probability of inflammation and pain in a method of prophylaxis.

“Topical” herein means application of the agent/compound to the skin or mucous membrane.

Accumulating evidence suggests that the peroxisome proliferator-activated receptors (PPARs), COX-2, and COX-2 derived prostaglandin (PG)E₂ participate in inflammation and cartilaginous destruction in OA and RA. (Chen, X. L. et al. 2003. Hosoya, T., et al. 2005. Dimmeler, S. 1996. Jang, J. H. & Surh, Y J. 2003). The peroxisome proliferator-activated receptors (PPARα, PPARβ/δ and PPARγ) are a family of ligand-activated transcription factors that up-regulate target genes containing the PPAR-responsive elements (PPAREs). Ample evidence also suggests that the PPAR isoforms and cognate ligands are differentially regulated in a tissue and stimulus dependent manner (Voehringer, D. W., et al. 2000. Lee, M. S. et al. 2003). For example, PPARα but not PPARγ activators inhibit COX-2 and PG expression in aortic cells. (Abulencia, J. P., et al. 2003), and PPARγ but not PPARα expression is modulated in IL-I β stimulated rat condrocytes (Voehringer, D. W., et al. 2000). The PPARγ ligand, 15d-PGD2, is reported to modulate COX-2 in epithelial and smooth muscle cells by gene induction and via a negative feedback loop in eptithelial and smooth muscle cells. (Yokota, H. et al. 2003. Abulencia, J. P., et al. 2003). In mouse macrophages, 15d-PGD2 can activate NF-E2 related factor 2 (Nrf2), the transactivator of the phase 2 detoxifying enzymes (Amin, A. R. et al. 1997).

Cells defend themselves against external and internal toxins by increasing the expression of antioxidant/detoxifying genes, the phase 2 enzymes. The phase 2 gene products modify electrophilic intermediates to render them less reactive and harmful as well as increasing the expression of genes that participate in the defensive arsenal. For example, the phase 2 gene, Glutathione (GSH) transferase is a phase 2 enzyme that conjugates hydrophobic electrophiles with GSH, attenuating the electrophile's damaging properties. Another phase 2 enzyme, Quinone Reductase (QR) promotes the electron transfer of quinones and by this reduction down-modulates their ability to deplete intracellular GSH. Other phase 2 enzymes such as UDP-glucuronosyltransferases and epoxide hydrolase modify potential reactive species facilitating their excretion. The induction of phase-2 enzymes is also accompanied by the up-regulation of GSH itself.

Phase 2 detoxifying enzymes share a common cis regulatory region, the antioxidant response element (ARE) and its cognate transactivator, NF-E2 related factor 2 (Nrf2). Nrf2 is a cytoplasmic protein but upon induction translocates to the nucleus, binds to other nuclear proteins and participates in phase 2 enzyme gene activation. Edible plants, the cruciferous vegetables such as broccoli, contain high concentrations of potent activators of phase 2 genes, the class of small molecules, the isothiocyanates, sulforaphane [(−)-1-isothiocyanato-4(R)-(methylsulfinyl)-butane] and its parent compound, the glucosinolates [β-thioglucoside N-hydroximinosulfate, also known as (Z)-(or cis)-N-hydroximinosulfate esters or S-glucopyranosyl thiohydroximates] (Fahey, J. W. et al. 2001, Fahey, J. W. et al. 2002). A representative listing of glucosinolates and isothiocyanates are found in Fahey, J. W. et al. 2001 the contents which are incorporated herein by reference. Representative glucosinolates and sulforaphane analogs are listed below.

TABLE 1
Chemical and common names of glucosinolates identified in higher plants.
Class assignements refer to structure based assignment of glucosinolates
to chemical classes.

| | | | |
|---|---|---|---|
| 1 | F | 3-Methoxycarbonylpropyl | Glucoerypestrin |
| 2 | I | 1-Acetyl-indol-1-ylmethyl | 1-acetyl-glucobrassiein |
| 3 | J | 4-(4′-O-Acetyl-α-L- | |
| | | rhamnopyranosyloxy)benzyl | |
| 4 | J | 2-(α-L-Arabinopyranosyloxy)- | |
| | | 2•phenylethyl | |
| 5 | H | 4-(Benzoyloxy)butyl | |
| 6 | H | 2-(Benzoyloxy)ethyl | |
| 7 | H | 2-Benzoyloxy-1-ethylethyl | Glucobenzaisaustricin |
| 8 | H | Benzoyloxymethyl | |
| 9 | H | 2-Benzoyloxy-1-methylethyl | Glucobenzosisymbrin |
| 10 | H | 3-(Benzoyloxy)propyl | Glucomalomiin |
| 11 | G | Benzyl | Glucotropaeolin |
| 12 | D | 3-Butenyl | Gluconapin |
| 13 | B | n-Butyl | |
| 14 | G | 3,4-Dihydroxybenzyl | Glucomatronalin |
| 15 | G | 3,4-Dimethoxybenzyl | |
| 16 | B | Ethyl | Glucolepidiin |
| 17 | E | 1-Ethyl-2-hydroxyethyl | Glucosisaustricin |
| 18 | D | 6-Heptanyl | |
| 19 | D | 5-Hexenyl | |
| 20 | B | n-Hexyl | |
| 21 | G | 2-Hydroxybenzyl | |
| 22 | G | 3-Hydroxybenzyl | Glucolepigramin |
| 23 | G | 4-Hydroxybenzyl | {Gluco}sinalbin |
| 24a | D | 2(R)-2-Hydroxy-3-butenyl | Progoitrin |
| 24b | D | 2(S)-2-Hydroxy-3-butenyl | Epiprogoitrin |
| 25 | E | 3-Hydroxybutyl | |
| 26 | E | 4-Hydroxybutyl | |
| 27 | E | 2-Hydroxyethyl | |
| 28 | I | 4-Hydroxyindol-3-ylmethyl | 4-Hydroxy- |
| | | | glucobrassiein |
| 29 | E | 2-Hydroxy-2-methylbutyl | Glucocleomin |
| 30 | E | 1-(Hydroxymethyl)propyl | |
| 31 | E | 2-Hydroxy-2-methylpropyl | Glucoconringiin |
| 32 | A | 3 -Hydroxy-6-(methylsulfinyl)hexyl | |
| 33 | A | 3-Hydroxy-5-(methylsulfinyl)pentyl | |
| 34 | A | 3-Hydroxy-6-(methylsulfonyl)hexyl | |
| 35 | A | 3-Hydroxy-5-(methylsulfonyl)pentyl | |
| 36 | A | 3-Hydroxy-6-(methylthio)hexyl | |
| 37 | A | 3-Hydroxy-5-(methylthio)pentyl | |
| 38 | D | 2-Hydroxy-4-pentenyl | {Gluco}napoleferin |
| 39 | E | 2-Hydroxypentyl | |
| 40 | G | 2(R)-Hydroxy-2•phenylethyl | Glucobarbarin |
| 41 | E | 2-Hydroxypropyl | |
| 42 | E | 3-Hydroxypropyl | |
| 43 | I | lndol-3-ylmethyl | Glucobrassicin |
| 44 | G | 2-Methoxybenzyl | |
| 45 | G | 3-Methoxybenzyl | Glucolimnathin |
| 46 | G | 4-Methoxybenzyl | Glueoaubrietin |
| 47 | I | 1-Methoxyindol-3-ylmethyl | Neoglucobrassicin |
| 48 | I | 4-Methoxyindol-3-ylmethyl | 4-Methoxy- |
| | | | glucobrassicin |
| 49 | G | 2-(4-Methoxyphenyl)-2.2- | |
| | | dimethylethyl [or | |
| | | 2 .2-dimethyl-2- | |
| | | (4-methoxyphenyl)ethyl)] | |
| 50 | G | 2-(4-Methoxyphenyl)-2- | |
| | | hydroxyethyl [or 2-hydroxy- | |
| | | 2-(4-methoxyphenylethyl)] | |
| 51 | B | Methyl | Glucocupparin |
| 52 | D | 3-Methyl-3-butenyl | |
| 53 | C | 1-Methybutyl | |
| 54 | C | 2-Methybutyl | |
| 55 | C | 3-Methybutyl | |
| 56 | C | 1-Methyethyl | Glucoputranjivin, |
| | | | isopropyl |
| 57 | E | 1-Methyl-2-hydroxyethyl | Glucosisymbrin |
| 58 | C | 3-Methylpentyl | |
| 59 | C | 4-Methylpentyl | |
| 60 | D | 2-Methyl-2-propenyl | |
| 61 | C | 1-Methylpropyl | Glucocochlearin, |
| | | | glucojiabutin sec- |
| | | | Butyl.2-Butyl |
| 62 | C | 2 -Methylpropyl | Isobutyl |
| 63 | A | 4-Methysulfinyl-3-butenyl | Glucoraphenin |
| 64 | A | 4-(Methylsulfinyl)butyl | Glucoraphanin |
| 65 | A | 10-(Methylsulfinyl)decyl | Glucocamelinin |
| 66 | A | 7-(Methylsulfinyl)heptyl | Glucoibarin |
| 67 | A | 6-(Methylsulfinyl)hexyl | Glucohesperin |
| 68 | A | 9-(Methylsulfinyl)nonyl | Glucoarabin |
| 69 | A | 8-(Methylsulfinyl)octyl | Glucohirsuitin |
| 70 | A | 7-Methylsulfinyl-3-oxoheptyl | |
| 71 | A | 8-Methylsulfinyl-3-oxooctyl | |
| 72 | A | 5-(Methylsulfinyl)pentyl | Glucoalyssin |
| 73 | A | 3-(Methylsulfinyl)propyl | Glucoiberin |
| 74 | A | 11-(Methylsulfinyl)undecyl | |
| 75 | A | 4-Methysulfonyl-3-butenyl | |
| 76 | A | 4-(Methylsulfonyl)butyl | Glucoerysolin |
| 77 | A | 10-(Methylsulfonyl)decyl | |
| 78 | A | 6-(Methylsulfonyl)hexyl | |
| 79 | A | 9-(Methylsulfonyl)nonyl | |
| 80 | A | 8-(Methylsulfonyl)octyl | |
| 81 | A | 5-(Methylsulfonyl)pentyl | |
| 82 | A | 3-(Methylsulfonyl)propyl | Glucocheirolin |
| 83 | A | 4-Methylthio-3-butenyl | Dehydroerucin |
| 84 | A | 4-(Methylthio)butyl | Glucoerucin |
| 85 | A | 10-(Methylthio)decyl | |
| 86 | A | 2-(Methylthio)ethyl | Glucoviorylin |
| 87 | A | 7-(Methylthio)heptyl | |
| 88 | A | 6-(Methylthio)hexyl | Glucolesquerellin |
| 89 | A | 9-(Methylthio)nonyl | |
| 90 | A | 7-Methylthio-3-oxoheptyl | |
| 91 | A | 6-Methylthio-3-oxohexyl | |
| 92 | A | 8-(Methylthio)octyl | |
| 93 | A | 8-Methylthio-3-oxooctyl | |
| 94 | A | 5-(Methylthio)pentyl | Glucoberteroin |
| 95 | A | 3-(Methylthio)propyl | Glucoiberverin |
| 96 | F | 4-Oxoheptyl | Glucocapangulin; |
| | | | glucopangulin |
| 97 | F | 5-Oxoheptyl | Gluconorcappasalin |
| 98 | F | 5-Oxooctyl | Glucocappasalin |
| 99 | F | 4-Oxopentyl or 3- | |
| | | (Methylcarbonyl)propyl | |
| 100 | D | 1-Pentenyl | |
| 101 | D | 4-Pentenyl | Glucobrassicanapin |
| 102 | B | n-Pentenyl | |
| 103 | G | Phenyl | |
| 104 | G | 4-Phenylbutyl | |
| 105 | G | 2-Phenylethyl | Gluconasturtiin: |
| | | | phenylethyl |
| 106 | G | 3-Phenylpropyl | |
| 107 | D | 2-Propenyl | Allyl. Sinigrin |
| 108 | B | n-Propyl | |
| 109 | J | 2-(α-L-Rhamnopyranosyloxy) | |
| | | benzyl | |
| 110 | J | 4-(α-L-Rhamnopyranosyloxy) | |
| | | benzyl | |
| 111 | J | 6-Sinapoyl-β-D-1-thioglycoside | |
| | | of 4-methylsulfinylbut-3-enyl | |
| 112 | I | 1-Sulfo-indol-3-ylmethyl | Glucobrassicin-1-sulfate |
| 113 | E | 4,5,6,7-Tetrahydroxydecyl | |
| 114 | G | 3,4,5-Trimethoxybenzyl | |
| 115^{a} | | “iso”-Heptyl | |
| 116^{a} | | “iso”-Hexyl | |
| 117^{b} | H | 5-(Benzoyloxy)pentyl | |
| 118^{b} | H | 6-(Benzoyloxy)hexyl | |
| 119^{c} | | 3-0-Apiosylglucomatronalin | |
| 120^{c} | | 3-0-Apiosylglucomatronalin 3,4- | |
| | | dimethoxybenzoyl ester | |

^{a}Structures unresolved; Grob and Matile (1980).
^{b}Added in proof; Haughn et al. (1991): identified in Arabidosis sp.
^{c}Larsen et al. (1992): however, the identification of these compounds references only unpublished work.

TABLE 2
Representative Sulforaphane analogs.

| | | CD |
|---|---|---|
| ID | STRUCTURE | (μM) |
| GHP 1001 | 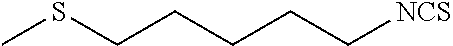 | 1.71 |
| GHP 1002 | 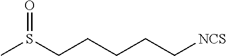 | 0.94 |
| GHP 1003 | 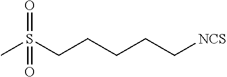 | 0.98 |
| GHP 1004 | 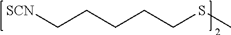 | 0.83 |
| GHP 1005 | 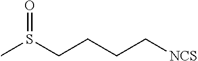 | 0.20 |
| GHP 1006 | 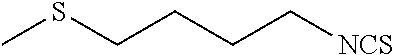 | 2.30 |
| GHP 1007 | 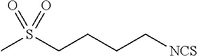 | 0.82 |
| GHP 1008 | 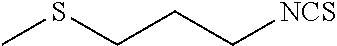 | 3.52 |
| GHP 1009 | 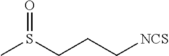 | 2.36 |
| GHP 1010 | 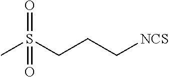 | 1.32 |
| GHP 1021 | 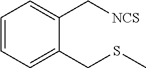 | 4.3 |
| GHP 1022 | 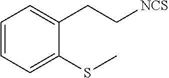 | 7.4 |
| GHP 1023 | 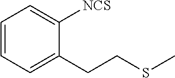 | 100 |
| GHP 1031 | 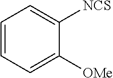 | 100 |
| GHP 1032 | 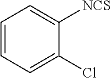 | 100 |
| GHP 1033 | 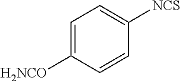 | 100 |
| GHP 1041 | 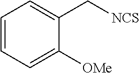 | 2.41 |
| GHP 1042 | 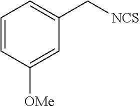 | 8.65 |
| GHP 1043 | 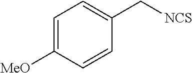 | 25 |
| GHP 1044 | 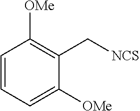 | 5.8 |
| GHP 1045 | 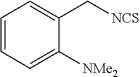 | 12.5 |
| GHP 1046 | 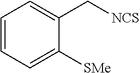 | 6.8 |
| GHP 1047 | 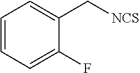 | 13.1 |
| GHP 1048 | 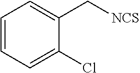 | 14.1 |
| GHP 1049 | 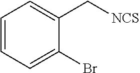 | 12.5 |
| GHP 1050 | 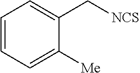 | 3.7 |
| GHP 1051 | 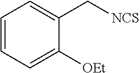 | 2.5 |
| GHP 1052 | 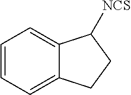 | 38.9 |
| GHP 1053 | 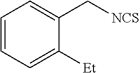 | 12.5 |
| GHP 1061 | 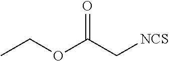 | 8.2 |
| GHP 1062 | 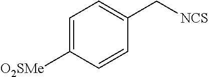 | 1.2 |
| GHP 1063 | 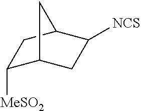 | 1.02 |
| GHP 1064 | 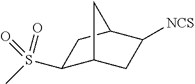 | 0.66 |
| GHP 1065 | 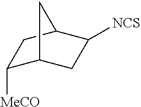 | 0.77 |
| GHP 1066 | 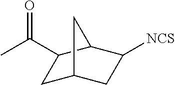 | 0.26 |
| GHP 1067 | 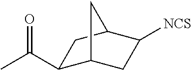 | 0.43 |
| GHP 1068 | 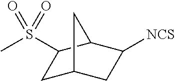 | 0.15 |
| GHP 1069 | 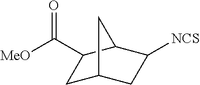 | 0.68 |
| GHP 1070 | 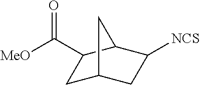 | 1.6 |
| GHP 1071 | 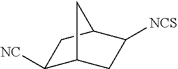 | 0.59 |
| GHP 1072 | 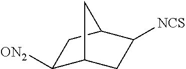 | 1.05 |
| GHF 1073 | 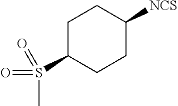 | 0.44 |
| GHP 1074 | 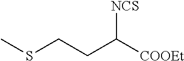 | 2.64 |
| GHP 1075 | 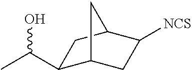 | 0.45 |
| GHP 1076 | 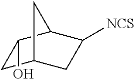 | 1.10 |
| GHP 1077 | 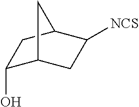 | 1.85 |
| GHP 1078 | 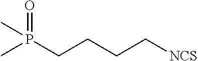 | 0.43 |
| GHP 1079 | 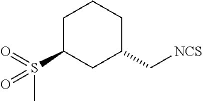 | 0.48 |
| GHP 1080 | 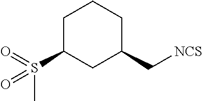 | 0.41 |
| GHP 1081 | 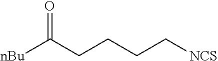 | 2.0 |
| GHP 1101 | 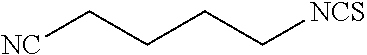 | 1.97 |
| GHP 1102 | 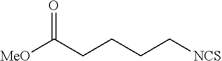 | 2.81 |
| GHP 1103 | 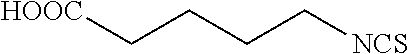 | 2.19 |
| GHP 1104 | 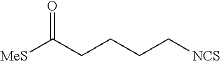 | 2.8 |
| GHP 1105 | 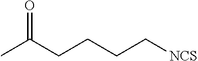 | 0.23 |
| GHP 1106 | 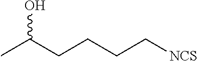 | 0.35 |

Fluid shear is a critical physiological stimulus that modulates intracellular signaling in a time, magnitude and phenotype dependent manner. Low laminar shear in human vessels tend to be atherogenic whereas high laminar shear tends to be atheroprotective. Exposure of human aortic endothelial cells to high laminar shear flow at 20 dynes (dyn)/cm2 (1 dyn=10 μN) induces expression of the Phase 2 genes. Moreover laminar shear flow potently inhibits apoptosis in growth factor-starved human umbilical vein endothelial cells (HUVECs) (Dimmeler, S. 1996). However, low intracellular GSH levels have been linked to mitochondrial depolarization and apoptosis in multiple cells lines (Jang, J. H. & Surh, Y. J. 2003. Voehringer, D. W., et al. 2000.

In marked contrast, extensive mechanical loading of cartilage producing both low hydrostatic pressure and high fluid (20 dynes (dyn)/cm²) shear results in irreversible chondrocyte apoptosis, matrix erosion, and osteoarthritis, whereas low shear (<5 dyn/cm²) is chondroprotective (Carter, D. R., et al. 2004. Lee, M. S., et al. 2003). The inventors have previously shown that high shear induces COX-2 expression in human chondrocytic cells through a c-Jun N-terminal kinase 2 (JNK2) dependent pathway (Abulencia, J. P. et al. 2003).

While not being bound by theory, this invention teaches that in human chondrocytic cells, shear stress induces COX-2 expression, suppresses phosphatidyl-inositol 3-kinase (PI3-K) activity, which represses Nrf2 mediated transcription of the phase 2 enzyme genes. This effect is attenuated with addition of phase 2 inducers and with COX-2 specific inhibitors. This invention also teaches the unexpected finding of negative feedback loops where COX-2 expression and inflammatory signaling is repressed by the downstream activity of PI3-K and/or the phase 2 enzymes.

While not being bound by theory, this invention also teaches that in addition to shear-stress induced down regulation of phase 2 gene expression, human chondrocytic cells (T/C-28a2) exposed to high shear stress for 48 hours also results in selective and significant down regulation of the PPARα mRNA isoform and increases markers of apoptosis (BAX and Caspase-9 precursors). Pre-treatment of chondrocytic cells with the COX-2 selective blockers significantly reversed the shear-mediated changes of PPARα. Pre-treatment with PPARα selective ligand abolishes shear induced down regulation of Nrf2, and phase 2 gene transcripts as well as the elevated apoptosis markers.

### EXAMPLE 1

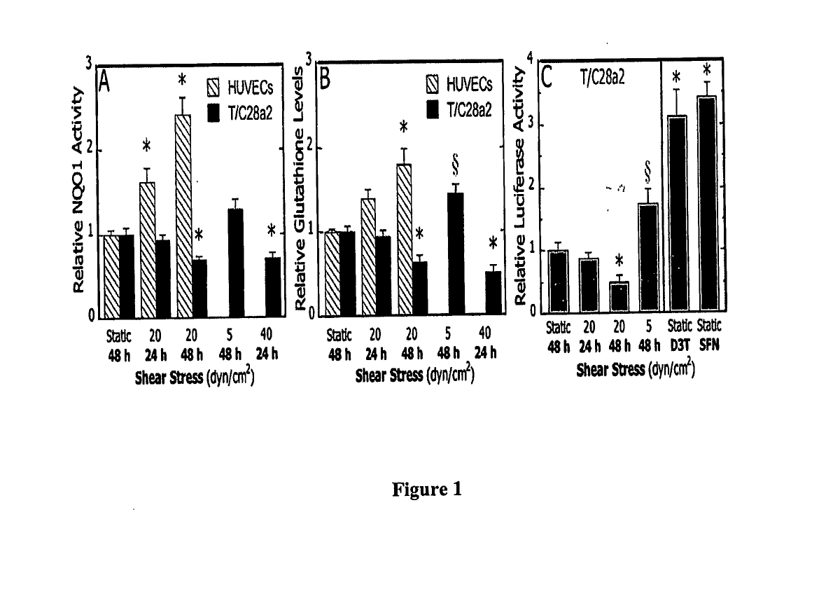

### FIG. 1 & Table 3 Below

Shear stress has a tissue specific effect on cellular anti-oxidant capacity. High shear induces mRNA expression of a battery of ARE-mediated genes in human umbilical vein endothelial cells (HUVEC), but decreases their expression in human T/C28a2 chondrocytic cells.

Cell Culture and Shear Stress Exposure: Human T/C28a2 chondrocytic cells were grown (37° C., 5% CO₂) in 1:1 Ham's F-12/DMEM (Biowhittaker) supplemented with 10% FBS. Prior to shear exposure, T/C28a2 cells were incubated for 24 hours in serum-free medium containing 1% Nutridoma-SP (Roche), a low-protein serum replacement that maintains chondrocyte phenotype. Primary HUVECs were cultured as described (Goldring, M. B., 2004). T/C28a2 cells were exposed to shear stress in media containing 1% Nutridoma by use of a parallel-plate flow chamber with a recirculating flow loop (37° C., 5% CO₂) (10). HUVECs were treated similarly by circulating media supplemented with 10% FBS.

Cell Viability, NQO1 activity, Glutathione levels, and Prostaglandin (PG)E₂ production: Cell viability was monitored with the MTT assay (Gao, X. et al. 2001). NQO1 activity and total GSH (oxidized and reduced) levels of cell lysates were determined in 96-well microtiter plates. PGE₂ levels were determined in media by the Prostaglandin E₂ monoclonal EIA kit (Cayman Chemical).

Transient Transfection and Plasmid Constructs: T/C28a2 cells were transfected with 10 μg of plasmid and 2 μg of control with Lipofectamine and Plus Reagent (Invitrogen). Cells were allowed to recover for 3 hours, incubated overnight in medium containing 1% Nutridoma, and exposed to the indicated treatments. Efficiency was assessed by flow cytometry with pEGFP-N2 (BD Biosciences). pCMV-mNrf2 and pNQO1/ARE-luc constructs were provided by N. Wakabayashi (Wakabayashi, N. et al. 2004). Igarashi, K., et al. 1994), and pBJ M-pl 10*-myc, pBJ M<<pl 10-UR, and pCG pi 10 wt constructs were provided by A. Kippel (Hu, Q., et al. 1995).

Promoter Activity Assay: T/C28a2 cells were transfected with 10 μg of pNQO1/ARE-luc and 1 μg each of pEGFP-N2 and pSV40-hRL2 (Promega) to normalize transfection efficiency. Firefly and Renilla luciferase activities were measured using the Dual-Luciferase Report Assay kit (Promega).

Intracellular Protein Staining and Western Blots: T/C28a2 cells were fixed in 1.0% formaldehyde for 10 min at 37° C., permeabilized in 90% methanol for 20 minutes on ice, and incubated at 25° C. for 10 min in blocking buffer (0.5% BSA). Specimens were then incubated with fluorophore-conjugated monoclonal antibodies specific for COX-I (COX-1/FITC) and COX-2 (COX-2/PE) (Cayman Chemical) or isotype controls (BD Biosciences) for 30 min, washed 2× in blocking buffer, and analyzed by flow cytometry. For Western blots, total cell lysates were subjected to SDS/PAGE, transferred to a membrane, and probed with caspase-9 and β-actin antibodies (Upstate).

Microarray Hybridization and Analysis: Cy-3- and Cy-5-labeled probes were mixed, dried, resuspended in hybridization buffer (50% formamide, 10×SSC, 0.2% SDS, COT-I DNA, Poly(A)-DNA), and denatured. The probes were added to microarray slides printed with a set of 32,448 or 39,936 expressed sequence tags (ESTs), allowed to hybridize at 42° C. overnight, and processed as described (Abulencia, J. P., et al. 2003. Hegde, P. et al. 2000). Expression ratios were derived using TIGR Spotfinder (Abulencia, J. P. et al. 2003, Hegde, P. et al. 2000). Differentially expressed genes were identified by Significance Analysis of Microarrays, and analyzed with the software TMEV (Abulencia, J. P. et al. 2003).

Quantitative Real-Time PCR (qRT-PCR) was used to verify DNA microarray data. Incorporation of SYBR Green into PCR products was monitored with the 7900HT detection system.

Exposure of primary HUVECs to 20 dyn/cm² increased the phase 2 enzyme, NQO1 protein activity and GSH levels in a time-dependent manner. In contrast to HUVECs, prolonged exposure (48 hours) of human chondrocytic cells, T/C28a2 to 20 dyn/cm² significantly decreased both NQO1 activity and GSH protein levels which correlates well with NQO1 and GCLR mRNA levels. Also, microarray analysis reveals that prolonged exposure (48 hours) of T/C28a2 cells to a shear level of 20 dyn/cm² results in a marked reduction in Nrf2 and phase 2 transcript expression, including NQO1, HO-I, GST and GCLR.

T/C28a2 cells transfected with NQO1/ARE-luciferase plasmid and exposed to 20 dyn/cm² for 48 hours resulted in a substantial reduction of the ARE-driven promoter activity. Addition of phase 2 enzyme transcription inducers, D3T (1,2-dithiole-3-thione) and SFN (sulforaphane), to the transfected cells increases the luciferase activity providing supporting evidence that ARE promoters are functionally intact.

TABLE 3
DNA microarray analysis of shear-induced mRNA response of chondrocytes. Data represent
microarray inentisity ratios (shear/static) of sheared (20 dyn/cm2, 48 hr) to paried static controls
of T/C28a2 cells. Paired treatments consisted of (*) no treatment; (†) 5 μM D3T (shear) and 0.1%
DMSO (static); and (‡) pBJ M*p110* (shear, 53% transfection efficiency) and pBJ-null (static).
Data represent mean ± SD (n = 5 − 8).

| Signaling Molecules of Interest | 48 h* | 5 μM D3T † | M*p110* ‡ |
|---|---|---|---|
| Protein Kinases & Transcription Factors | 20 dyn/cm² | 20 dyn/cm² | 20 dyn/cm² |
| JNK2 (c-jun N-terminal kinase 2) | 7.2 ± 0.5 | 1.6 ± 0.1 | 1.3 ± 0.1 |
| PI3K (phosphoinositol kinase 3) | 0.3 ± 0.1 | 0.8 ± 0.2 | 3.9 ± 0.7 |
| Transcription Factor AP-1 (c-jun) | 4.0 ± 0.4 | 1.4 ± 0.1 | 1.0 ± 0.2 |
| Nrf2 (NF-E2 related factor 2) | 0.7 ± 0.1 | 1.3 ± 0.1 | 0.9 ± 0.1 |
| Catalytic and Effector Proteins | | | |
| COX-2 (cyclooxygenase-2) | 3.5 ± 0.2 | 1.3 ± 0.2 | 1.7 ± 0.2 |
| COX-1 (cyclooxygenase-1) | 1.1 ± 0.1 | 1.3 ± 0.1 | 0.8 ± 0.2 |
| Caspase-9 precursor | 1.5 ± 0.1 | 0.8 ± 0.1 | 0.8 ± 0.2 |
| Phase 2 and Antioxidative Proteins | | | |
| NQO1 (NAD(P)H:quinone reductase-1) | 0.7 ± 0.1 | 1.3 ± 0.1 | 1.4 ± 0.1 |
| HO-1 (Heme Oxygenase-1) | 0.6 ± 0.1 | 1.2 ± 0.1 | 1.9 ± 0.2 |
| HO-2 (Heme Oxygenase-2) | 1.1 ± 0.1 | 0.9 ± 0.1 | 1.1 ± 0.1 |
| GST μ1 (glutathione S-transferase) | 0.5 ± 0.1 | 1.6 ± 0.1 | 1.3 ± 0.1 |
| UDP-glucuronosyltransferase | 0.7 ± 0.1 | 1.0 ± 0.1 | 1.8 ± 0.2 |
| GCLR (γ-glutamylcysteine ligase) | 0.5 ± 0.1 | 1.6 ± 0.1 | 2.0 ± 0.1 |
| Glutathione reductase | 0.4 ± 0.1 | 2.8 ± 0.1 | 2.4 ± 0.4 |

### EXAMPLE 2

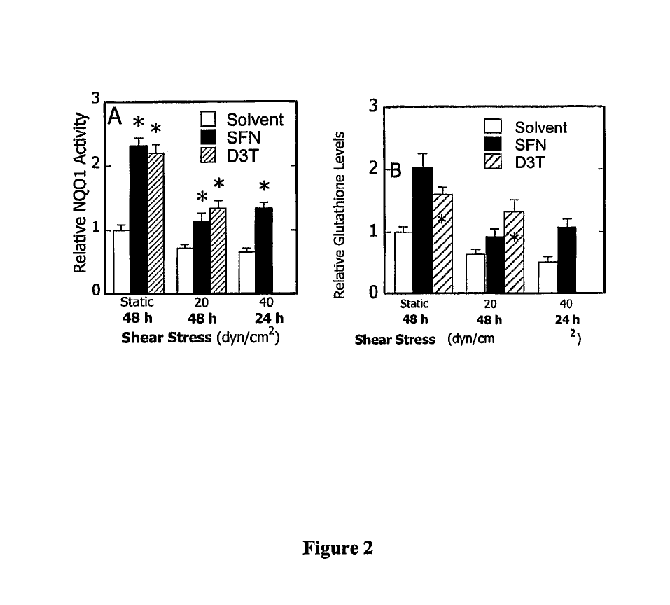

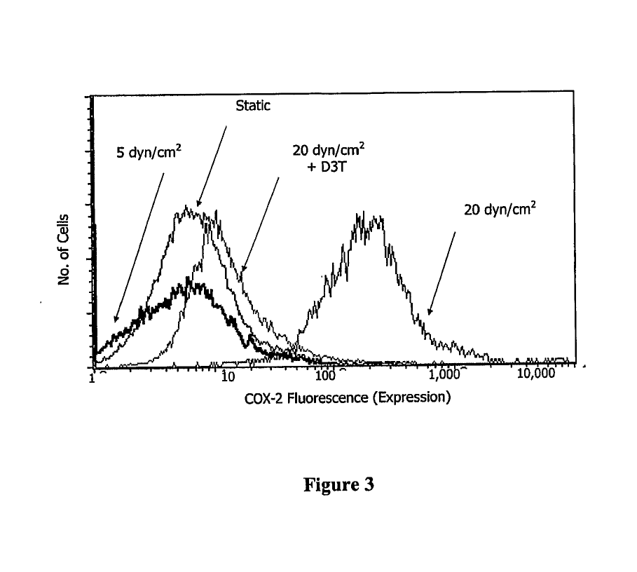

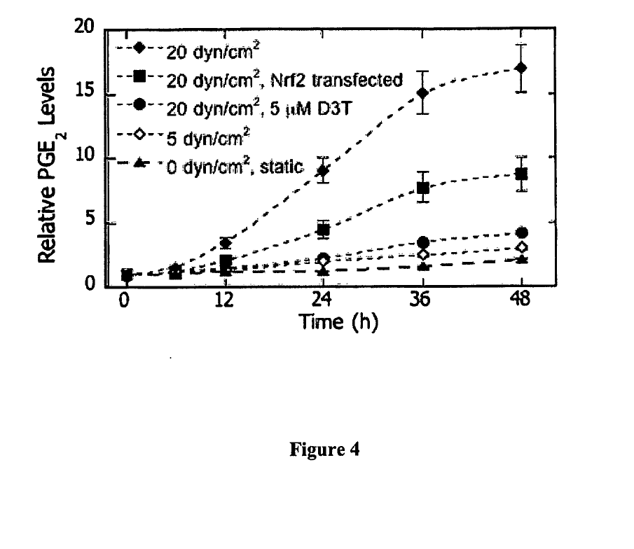

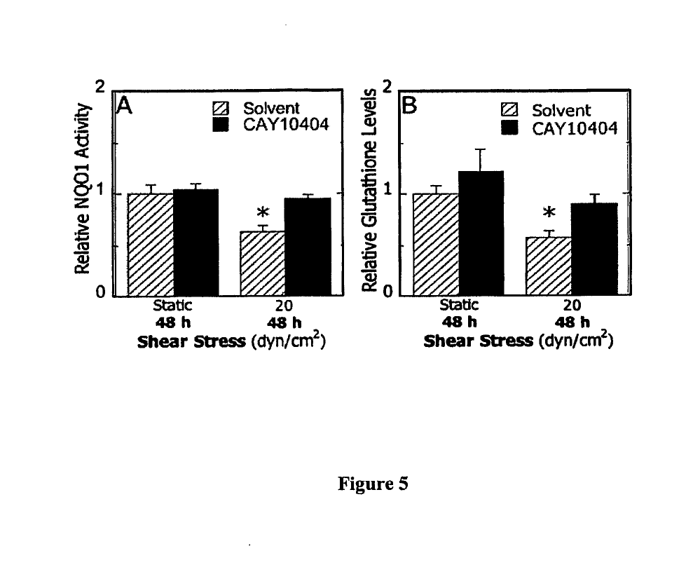

### FIGS. 2, 3, 4, 5

The shear mediated reduction in antioxidant capacity and corresponding pro-inflammatory state (increased COX-2 and PGE₂ levels), in chondrocytes is reversed with phase 2 inducers and COX-2 specific inhibitors.

Cell culture and shear stress exposure, cell viability, promoter activity and intracellular protein staining and western blots, microarray analysis and qRT-PCR assays were carried out in example 1. Treatment of high shear exposed T/C28a2 cells with the potent phase 2 enzyme inhibitor found in edible plants, sulforaphane, SFN (1.25 μM) abolished the shear-induced suppression of the phase 2 enzyme NQO1 activity and interacellular GSH levels. Furthermore, 1,2-dithiole-3-thione, D3T (5 μM), a specific inducer of the Nrf2/ARE pathway was likewise effective in suppressing the shear-mediated reduction of phase 2 enzyme activity.

Application of high shear, at least 20 dyn/cm2 to chondrocytes increases COX-2 transcript levels (Table 3) and COX-2 dependent PGE₂ production in T/C28a2 cells in a time-dependent fashion. D3T nearly abrogated both COX-2 protein expression and PGE₂ production in chondrocytes subjected to high shear. Transfection with pCMV plasmid containing murine Nrf2 substantially reduced the PGE₂ production (53%), consistent with 33% transfection efficiency of pCMV-mNrf2.

Furthermore, Addition of the highly selective COX-2 inhibitor, CAY10404 (6.75 μM) for 2 hours before and during shear exposure (20 dyn/cm² for 48 hours), reduced the shear-induced down-regulation of NQO1 activity and intracellular GSH levels.

### EXAMPLE 3

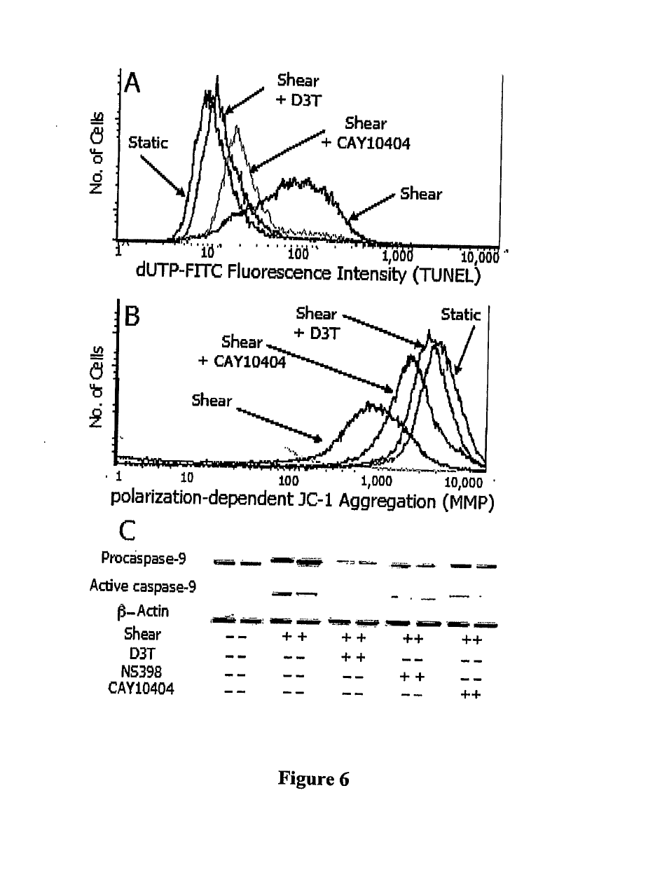

### FIG. 6 & Table 3

Shear induced chondrocyte apoptosis is suppressed by phase 2 inducers and COX-2 specific inhibitors.

Cell culture and shear stress exposure, cell viability, promoter activity and intracellular protein staining and western blots, microarray analysis and qRT-PCR assays were carried out as in example 1.

DNA Fragmentation and Mitochondrial Depolarization: For DNA fragmentation, cells were fixed in 4% paraformaldehyde for 1 hour at 25° C., washed 2× in PBS, and permeabilized briefly in 0.1% Triton-X100/0.1% sodium citrate on ice. Subsequently, cells were washed 2× in PBS, labeled using the In Situ Cell Death Kit (Roche), and analyzed by flow cytometry. To quantify Mitochondrial Membrane Potential (MMP), cells were labeled using the MitoProbe JC-I Kit (Molecular Probes).

Microscopic inspection of shear-stimulated (20 dyn/cm², 48 h) T/C28a2 cells showed cell shrinkage and membrane blebbing, providing morphological evidence of apoptosis. Moreover, transcriptional profiling (Table 3) revealed increased expression of procaspase-9 mRNA, an apoptotic effector molecule activated by mitochondrial depolarization, indicating the onset of apoptosis. We then monitored the effect of shear on apoptosis by measuring DNA fragmentation (TUNEL) and mitochondrial membrane depolarization (MMP). The presence of D3T (5 μM) essentially abrogated shear-induced apoptosis, whereas treatment with the COX-2 specific inhibitors CAY10404 (6.75 μM) and NS398 (30 μM) resulted in a marked reduction in apoptosis markers. Additionally, the role of caspase-9 in shear-mediated apoptosis was determined by immunoblot analysis, which revealed that high shear (20 dyn/cm²) increased the expression of both the proform (46 kDa) and active form (34 kDa) of caspase-9, whereas treatment with D3T or COX-2 selective inhibitors substantially reduced expression.

### EXAMPLE 4

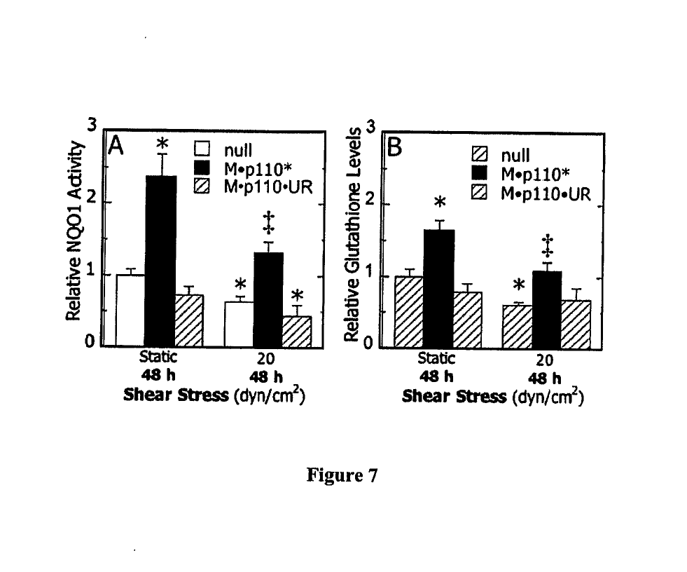

### FIG. 7 & Table 3

High shear represses PI3-K activity that down-regulates phase 2 enzymes and increases apoptosis in chondrocytes.

Cell culture and shear stress exposure, cell viability, promoter activity assay, microarray analysis were carried out as in example 1.

To identify potential signaling partners involved in the down regulation of phase 2 genes in shear-activated chondrocytes, differentially expressed genes were clustered using Support Trees. Analysis established that transcriptional regulation of PI3K (p85) paralleled that of Nrf2 and phase 2 genes, indicating that PI3K may be involved in the shear-mediated repression of ARE-regulated transcriptional activity and the onset of apoptosis. To examine the role of PI3K in this signaling cascade, T/C28a2 cells were transfected with a constitutively active PI3K mutant, M*p110* and exposed to shear. This intervention prevented the shear-mediated suppression of Nrf2, phase 2 genes and the induction of procaspase-9 (Table 3). Similarly, constitutively active PI3K was sufficient to enhance NQO1 activity and GSH levels in static cultures, and ablate their downregulation in sheared chondrocytes. Intriguingly, shear-induced COX-2 mRNA expression was markedly suppressed in chondrocytes transfected with the constitutively active form of PI3K (Table 3) but not the wild-type construct. PPARα

### EXAMPLE 4A

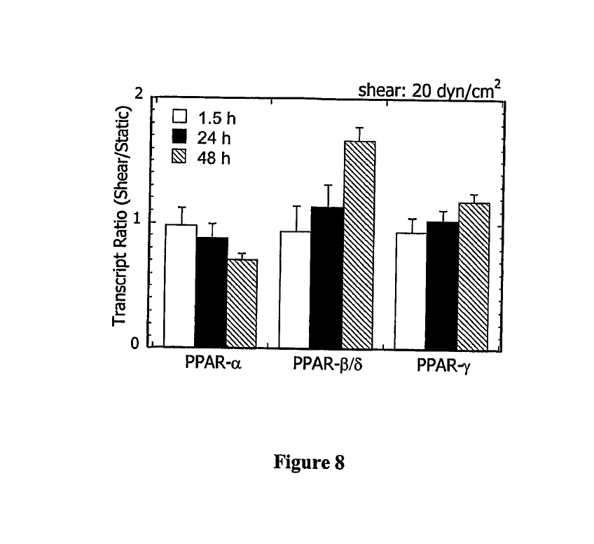

### FIG. 8

Exposure to high shear differentially regulates the PPAR isoforms.

Cell Culture and shear stress exposure, microarray hybridization and analysis and Quantitative Real-Time PCR (qRT-PCR) were carried out as in example 1. Exposure of human T/C-28a2 chondrocytes to a shear stress level of 20 dyn/cm² for 48 hours results in a selective and significant down regulation of PPARα mRNA expression and concomitant upregulation of PPARβ/δ mRNA synthesis, while leaving intact PPARγ transcript levels.

### EXAMPLE 5

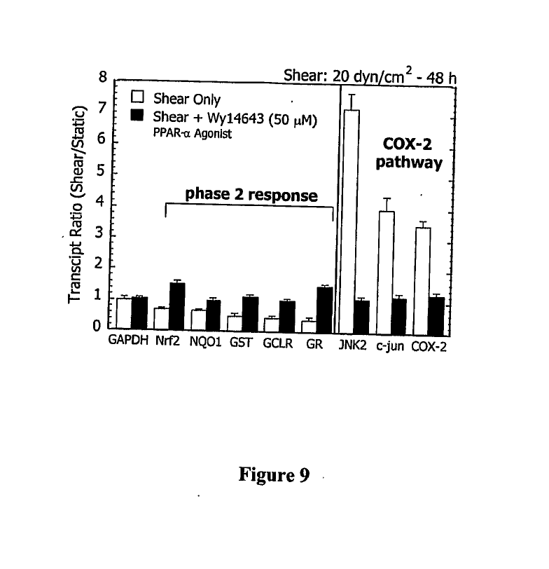

### FIG. 9 & Table 4 Below

PPARα ligand abolished the shear-induced down-regulation of the mRNA levels of Nrf2, phase 2 genes, apoptosis and shear induced upregulation of COX2, c-jun, and JNK2.

Cell Culture and shear stress exposure, microarray hybridization and analysis and quantitative real-time PCR (qRT-PCR) were carried out as in example 1. T/C-28a2 chondrocytes were pre-treated with a specific PPARα ligand, Wy14643 (10 μM) for 2 hours before being subjected to a shear stress level of 20 dyn/cm² for 48 hours in the presence of Wy14643. This pharmacological intervention abolished the shear-induced down regulation of the mRNA levels of Nrf2 and phase 2 genes and apoptosis as evidenced by the abrogation of shear-mediated changes of Bcl-w and pro-caspase-9 mRNA expression.

TABLE 4
Effects of a PPARα specific ligand on mRNA transcript
ratios (shear/static) in chondrocytes. Human T/C-28a2 cells,
pre-treated with either a PPARα specific ligand
(Wy14643; 10 μM) or solvent (none) were sheared at
20 dyn/cm² for 48 h. All values, obtained by qRT-PCR,
represent transcript ratios for sheared to
paired static controls. Data are mean + range (n = 2).
N/A: Not Available.

| | qRT-PCR Transcript | |
|---|---|---|
| | Ratio (Shear/Static) | |
| Molecular Signaling Families | Pharmacological Agent | |
| Transcription Factor | PPARα | None |
| Nrf2 (NF-E2 related factor 2) | 1.6 ± 0.1 | 0.7 ± 0.1 |
| Arachidonate | | |
| Metabolism and Signaling | | |
| iNOS (inducible Nitric | 1.0 ± 0.2 | 1.8 ± 0.1 |
| Oxide Synthase II) | | |
| COX-2 (cyclooxygenase-2) | 1.3 ± 0.2 | 4.4 ± 0.4 |
| PGE₂ Receptor Subtype EP4 | 0.6 ± 0.1 | 2.3 ± 0.2 |
| PPARα | 1.1 ± 0.2 | 0.8 ± 0.1 |
| Phase 2 Detoxifying & | | |
| Antioxidant Enzymes | | |
| NQO1 (NAD(P)H: | 1.0 ± 0.1 | 0.6 ± 0.1 |
| quinone oxidoreductase-1) | | |
| GST μ1 (glutathione-S- | 1.3 ± 0.2 | 0.4 ± 0.2 |
| transferase class μ1) | | |
| GCLR (glutamate-cysteine ligase) | 1.2 ± 0.2 | 0.2 ± 0.1 |
| Apoptosis-related | | |
| or Catabolic Gene(s) | | |
| Bcl-w (Bcl-2-like protein 2) | 1.0 ± 0.1 | 0.6 ± 0.1 |
| Caspase-9 precursor | 1.0 ± 0.1 | 1.6 ± 0.2 |
| MMP-9 (matrix metalloproteinase-9) | 1.3 ± 0.1 | 2.5 ± 0.3 |

### EXAMPLE 6

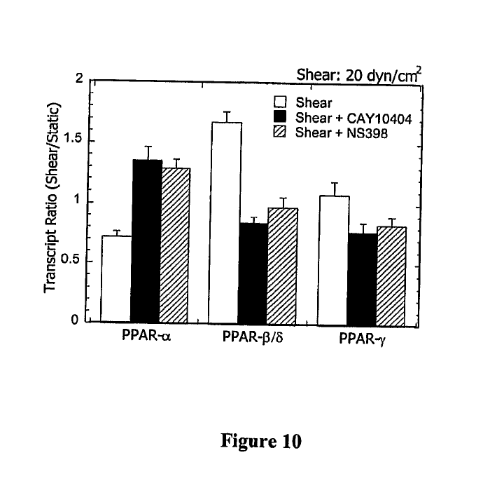

### FIG. 10 & Table 4

PPARα ligand interferes with COX-2 expression.

Cell Culture and shear stress exposure, microarray hybridization and analysis and Quantitative Real-Time PCR (qRT-PCR) were carried out as in example 1. T/C-28a2 chondrocytes were pre-treated with a specific PPARα ligand, Wy14643 (10 μM) for 2 hours before being subjected to a shear stress level of 20 dyn/cm² for 48 hours in the presence of Wy14643. Inspection of cDNA microarray data reveals that the selective PPARα ligand WyI 4643 nearly abrogates the shear-mediated up-regulation of JNK2 and c-Jun mRNA levels.

### REFERENCES

1. Amin, A. R., Attur, M., Patel, R. N., Thakker, G. D., Marshall, P. J., Rediske, J., Stuchin, S. A., Patel, I. R. & Abramson, S. B. (1997) J. Clin. Invest. 99, 1231-1237.
2. Abulencia, J, P., Gaspard, R., Healy, Z. R., Gaarde, W. A., Quackenbush, J. & Konstantopoulos, K. (2003) J. Biol. Chem. 278, 28388-28394.
3. Carter, D. R., Beaupre, G. S., Wong, M., Smith, R. L., Andriacchi, T. P., Schurman, D. J. & Smith, R. L. (2004) Clin. Orthop. 427S, S69-77.
4. Chen, X. L., Varner, S. E., Rao, A. S., Grey, J. Y., Thomas, S., Cook, C. K., Wasserman, M. A., Medford, R. M., Jaiswal, A. K. & Kunsch, C. (2003) J. Biol. Chem. 278, 703-711.
5. Churchill's Medical Dictoinary, Ruth Koenigsberg et al. (eds), Churchill Livingstone Inc., 1989.
6. Cunard, Robyn. (2005) Current Opinion in Investigational Drugs 6 (5), 467-472.
7. Dimmeler, S., Haendeler, J., Rippmann, V., Nehls, M. & Zeiher, A. M. (1996) FEBS Lett. 399, 71-74.
8. Fahey, J. W., Zalcmann, A. T., & Talaly, P. (2001) Phytochemistry 56, 5-51.
9. Fahey, J. W., Haristoy, X., Dolan, P. M., Kensler, T. W., Scholtus, I., Stephenson, K. K., Talalay, P. & Lozniewski, A. (2002) Proc. Natl. Acad. Sci. USA 99, 7610-7615.
10. Goldring, M. B. (2004) Clinical Orthopaedics and Related Research. No. 427S, pp S37-S46.
11. Goldring, M. B. (2004) Methods Mol. Med. 100, 37-52.
12. Gao, X., Dinkova-Kostova, A. T. & Talalay, P. (2001) Proc. Natl. Acad. Sci. USA 98, 15221-15226.
13. Hegde, P., Qi, R., Abernathy, K., Gay, C, Dharap, S., Gaspard, R., Hughes, J. E., Snesrud, E., Lee, N. & Quackenbush, J. (2000) Biotechniques 29, 548-556.
14. Hosoya, T., Maruyama, A., Kang, M. I., Kawatani, Y., Shibata, T., Uchida, K., Itoh, K. & Yamamoto, M. (2005) J. Biol. Chem. 280, 27244-27250.
15. Hu, Q., Klippel, A., Muslin, A. J., Fantl, W. J. & Williams, L. T. (1995) Science 268, 100-102.
16. Igarashi, K., Kataoka, K., Itoh, K., Hayashi, N., Nishizawa, M. & Yamamoto, M. (1994) Nature 367, 568-572.
17. Jong, J. H. & Surh, Y. J. (2003) Biochem. Pharmacol. 66, 1371-1379.
18. Kiefer, W., and Dannhardt, G. (2004) Current Medicinal Chemistry, 11, 3147-3161.
19. Kota, B. P. et al. (2005) Pharmacological Research 51, 85-94.
20. Lee, M. S., Trindade, M. C. D., Ikenoue, T., Goodman, S. B., Schurman, D. J. & Smith, R. L. (2003) J. Cell. Biochem. 90, 80-86.
21. Lovet-Racke, A. E. (2004) Journal of Immunology. 172, 5790-5798.
22. Voehringer, D. W., Hirschberg, D. L., Xiao, J., Lu, Q., Roederer, M., Lock, C. B., Herzenberg, L. A. & Steinman, L. (2000) Proc. Natl. Acad. Sci. USA 97, 2680-2685.
23. Wakabayashi, N., Dinkova-Kostova, A. T., Holtzclaw, W. D., Kang, M.-L, Kobayashi, A., Yamamoto, M., Kensler, T. W. & Talalay, P. (2004) Proc. Natl. Acad. Sci. USA 101, 2040-2045.
24. Yokota, H., Goldring, M. B. & Sun, H. B. (2003) J. Biol. Chem. 278, 47275-47280

1.-70. (canceled)

71. A topical dermal lotion, cream foam or gel suitable for use in treating joint or cartilaginous inflammation or pain due to mechanical stress comprising a therapeutically effective amount of a selective PPARα agonist and a phase 2 gene activator and one or more additives and adjuvants in an amount effective to treat joint or cartilaginous inflammation or pain in a subject in need thereof.

72. The composition of claim 71, wherein said selective PPARα is selected from the group consisting of Wy14643, clofibrate, fenofibrate, 8(S) Hydroxy-(5Z,9E,11Z,14Z)-eicosatetraenoic acid (8(S)-HETE), leukotriene B4 (LTB4), tetradecythioacetic acid (TTA), GW 9578, and GW 7647.

73. The composition of claim 71, wherein said phase 2 gene activator comprises a glucosinolate or isothyocyanate.

74. The composition of claim 73, wherein said isothiocyanate is sulforaphane and/or one or more of said sulforaphane analogs listed as follows:

| | |
|---|---|
| GHP 1001 | 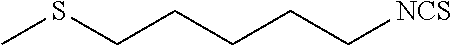 |
| GHP 1002 | 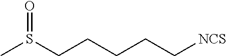 |
| GHP 1003 | 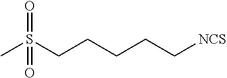 |
| GHP 1004 | 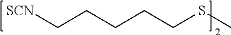 |
| GHP 1005 | 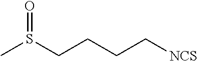 |
| GHP 1006 | 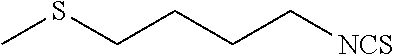 |
| GHP 1007 | 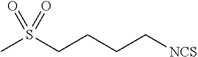 |
| GHP 1008 | 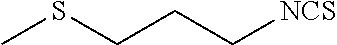 |
| GHP 1009 | 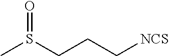 |
| GHP 1010 | 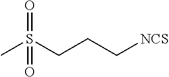 |
| GHP 1021 | 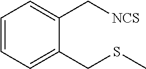 |
| GHP 1022 | 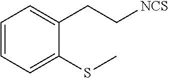 |
| GHP 1023 | 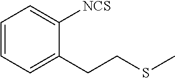 |
| GHP 1031 | 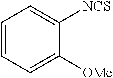 |
| GHP 1032 | 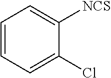 |
| GHP 1033 | 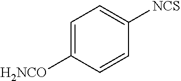 |
| GHP 1041 | 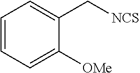 |
| GHP 1042 | 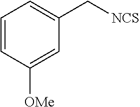 |
| GHP 1043 | 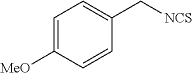 |
| GHP 1044 | 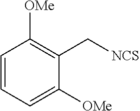 |
| GHP 1045 | 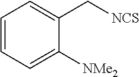 |
| GHP 1046 | 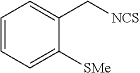 |
| GHP 1047 | 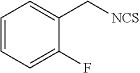 |
| GHP 1048 | 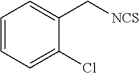 |
| GHP 1049 | 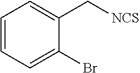 |
| GHP 1050 | 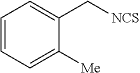 |
| GHP 1051 | 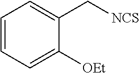 |
| GHP 1052 | 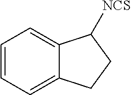 |
| GHP 1053 | 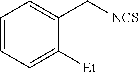 |
| GHP 1061 | 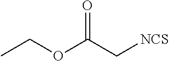 |
| GHP 1062 | 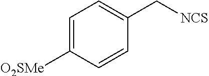 |
| GHP 1063 | 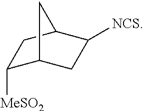 |
| GHP 1064 | 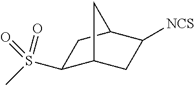 |
| GHP 1065 | 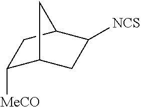 |
| GHP 1066 | 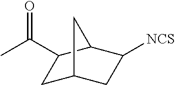 |
| GHP 1067 | 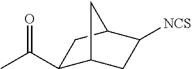 |
| GHP 1068 | 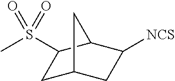 |
| GHP 1069 | 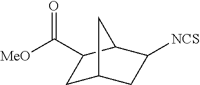 |
| GHP 1070 | 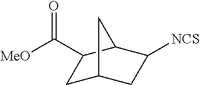 |
| GHP 1071 | 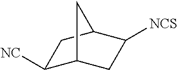 |
| GHP 1072 | 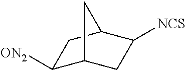 |
| GHP 1073 | 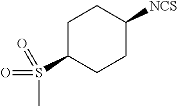 |
| GHP 1074 | 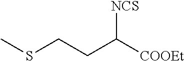 |
| GHP 1075 | 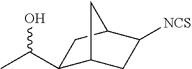 |
| GHP 1076 | 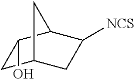 |
| GHP 1077 | 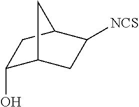 |
| GHP 1078 | 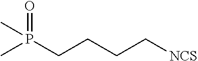 |
| GHP 1079 | 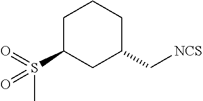 |
| GHP 1080 | 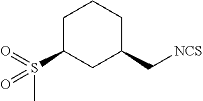 |
| GHP 1081 | 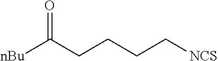 |
| GHP 1101 | 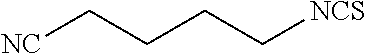 |
| GHP 1102 | 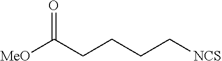 |
| GHP 1103 | 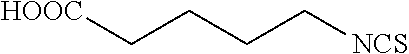 |
| GHP 1104 | 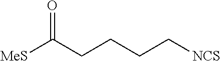 |
| GHP 1105 | 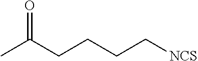 |
| GHP 1106 | 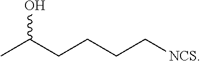 |

75. The composition of claim 71, wherein the joint or cartilaginous inflammation or pain is due to osteoarthritis.

76. A method for treating or preventing joint or cartilaginous inflammation or pain in a mammal due to mechanical stress comprising administering a therapeutically effective amount of the composition of claim 71 to the mammal.

77. The method of claim 76, wherein 0.05-150 mg/kg/day of said selective PPARα agonist of claim 1 is administered.

78. The method of claim 77, wherein said phase 2 gene activator comprises a glucosinolate or isothyocyanate.

79. The method of claim 78, wherein 1-50 mg/kg/day of said glucosinolate is administered.

80. The method of claim 78, wherein said isothiocyanate is sulforaphane and/or one or more of said sulforaphane analogs listed as follows:

| | |
|---|---|
| GHP 1001 | 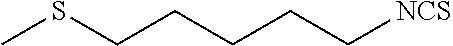 |
| GHP 1002 | 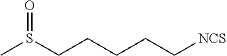 |
| GHP 1003 | 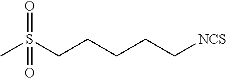 |
| GHP 1004 | 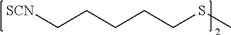 |
| GHP 1005 | 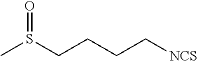 |
| GHP 1006 | 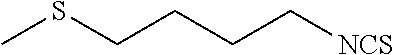 |
| GHP 1007 | 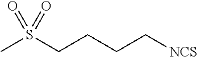 |
| GHP 1008 | 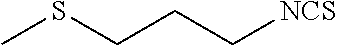 |
| GHP 1009 | 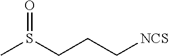 |
| GHP 1010 | 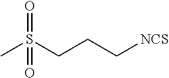 |
| GHP 1021 | 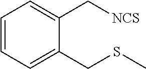 |
| GHP 1022 | 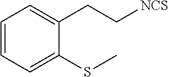 |
| GHP 1023 | 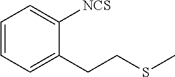 |
| GHP 1031 | 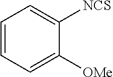 |
| GHP 1032 | 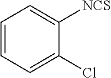 |
| GHP 1033 | 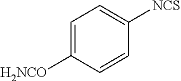 |
| GHP 1041 | 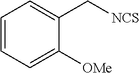 |
| GHP 1042 | 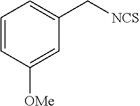 |
| GHP 1043 | 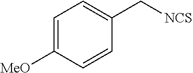 |
| GHP 1044 | 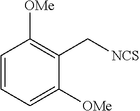 |
| GHP 1045 | 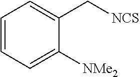 |
| GHP 1046 | 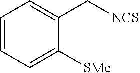 |
| GHP 1047 | 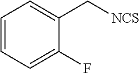 |
| GHP 1048 | 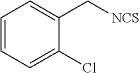 |
| GHP 1049 | 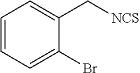 |
| GHP 1050 | 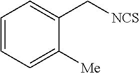 |
| GHP 1051 | 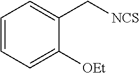 |
| GHP 1052 | 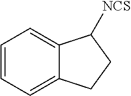 |
| GHP 1053 | 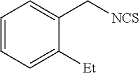 |
| GHP 1061 | 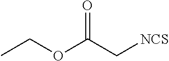 |
| GHP 1062 | 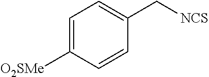 |
| GHP 1063 | 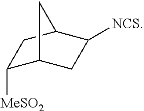 |
| GHP 1064 | 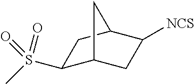 |
| GHP 1065 | 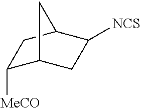 |
| GHP 1066 | 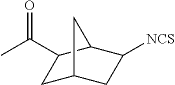 |
| GHP 1067 | 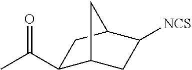 |
| GHP 1068 | 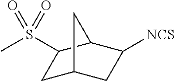 |
| GHP 1069 | 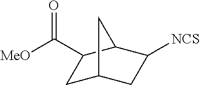 |
| GHP 1070 | 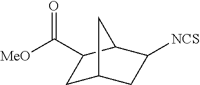 |
| GHP 1071 | 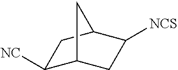 |
| GHP 1072 | 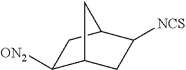 |
| GHP 1073 | 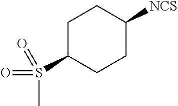 |
| GHP 1074 | 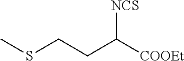 |
| GHP 1075 | 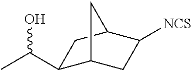 |
| GHP 1076 | 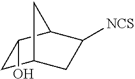 |
| GHP 1077 | 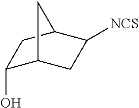 |
| GHP 1078 | 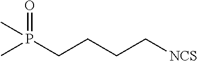 |
| GHP 1079 | 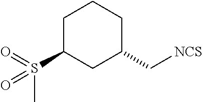 |
| GHP 1080 | 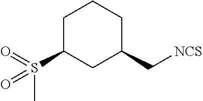 |
| GHP 1081 | 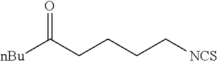 |
| GHP 1101 | 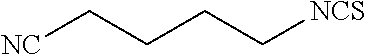 |
| GHP 1102 | 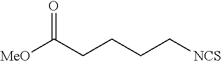 |
| GHP 1103 | 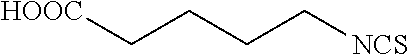 |
| GHP 1104 | 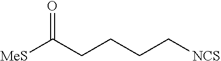 |
| GHP 1105 | 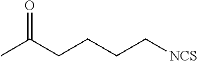 |
| GHP 1106 | 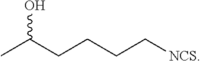 |

81. The method of claim 76, wherein said administration is topical.

82. The method according to claim 76, wherein said joint is a hip, a knee, an ankle, a shoulder, an elbow, a wrist or a joint of a foot, a joint of a hand, or a joint of the spine.

83. The method according to claim 76, wherein said joint is the temporomandibular joint.

84. The method of claim 76, wherein the joint or cartilaginous inflammation or pain is due to osteoarthritis.
